SEQUENCE LISTING (9 sequences)

SEQ ID NO: 1 - DNA, 13237 bp, Methylococcus capsulatus
  misc_feature 1..13237: note = pBZ34
  source 1..13237: mol_type = genomic DNA
ggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttttgacggctagctcagtcctagggataatgctagcaccagcctcgagggaaaccacgtaagctccggcgtttaaacacccataacagatacggactttctcaaaggagagttatcagtgaaaatccgcccgttacatgaccgtgtcatcatcaaacgcttggaagaagagcgtacctcggcgggcgggattgtcattccagatagcgcagctgaaaaaccgatgcgtggtgaaatcctggcagtgggcaatggaaaagtgcttgataatggagaggtacgtgctttacaggtgaaagtgggtgataaagtgctctttgggaaatacgcgggtacggaggttaaagtagatggggaagatgttgttgtcatgcgtgaagatgacattctggctgtgttagaatcttaatccgcgcacgacactgaacatacgaatttaaggaataaagataatggcgaaagaagttgtgtatcgtggtagtgcgcgccagcgtatgatgcagggtattgaaattctcgctcgcgccgctattccaacgctgggggcaaccggcccgagcgtcatgattcaacatcgcgccgatggtctgccacccatttctacacgcgatggcgttaccgtagcgaattctattgttttaaaagaccgtgtcgcgaacctgggtgcccgcctgctgcgcgacgtagccggtacaatgagccgtgaagccggcgacggcacgacgactgcgatcgtattggcccgccacatcgcccgtgagatgtttaaatcgctggccgtgggtgcagatccgatcgcgctgaaacgtggtatcgatcgcgccgttgctcgtgtgtccgaagatattggggcgcgtgcgtggcgtggcgataaagaaagcgtgatcctgggtgtcgctgctgtggcgacgaaaggcgaaccgggcgttggccgtctgctgctggaggctctcgatgcagtgggtgttcacggtgccgtttctatcgaactgggccaacgtcgtgaagatctgctggacgtcgtcgatggctatcgctgggaaaaaggttatttatctccctactttgtcacggaccgtgcccgcgaactcgcggaactggaggatgtctacctgctcatgaccgaccgcgaagtggttgacttcatcgaccttgtacctctgctggaggccgtgacggaagcaggaggctccctgctgattgccgcggatcgtgtgcacgaaaaggccttagcggggctgcttctgaatcacgtgcgcggtgtcttcaaggccgtggccgtaaccgctccgggttttggcgacaaacgcccgaaccgtttacttgacctggccgcgttaaccggcggtcgtgccgtgctcgaagctcaaggcgaccgtctggaccgtgttaccctcgcggatctgggccgtgtgcgccgtgccgtggtgtcggcagatgataccgcgctgcttggcatcccgggcaccgaagctagccgtgcacgcctcgaaggtctgcgtttagaagcagagcagtaccgtgcgctgaaaccagggcagggttctgccaccgggcgcctgcacgaacttgaagaaattgaagcgcgcattgtgggtctgtccggaaagagcgccgtttatcgcgtcggaggtgtgaccgatgtggaaatgaaagagcgcatggttcgcatcgaaaacgcttaccgttcggtggtaagtgcgctggaggaaggcgtgctccctggcggtggtgtcggctttctgggtagtatgccggtgcttgcggaattggaggcccgcgacgcagatgaagctcgcgggattgggattgtacgcagcgccttaacggagcctcttcgtattatcggcgaaaatagtggcttgagcggtgaagccgttgttgccaaagtcatggatcatgccaacccgggatggggttacgaccaggagtctggctctttttgcgacctgcatgcgcgtgggatctgggatgctgctaaagtgttacgtctcgcgttggagaaggcagcctctgttgctgggacctttctgacaaccgaagctgttgttctcgaaattccggatacagatgcgttcgcagggttcagtgcagaatgggctgccgccacgcgcgaagatccgcgcgtatgagtttaaacgcggccgcaatttgaacgcacccataacagatacggactttctcaaaggagagttatcaatgaatattcgtccattgcatgatcgcgtgatcgtcaagcgtaaagaagttgaaactaaatctgctggcggcatcgttctgaccggctctgcagcggctaaatccacccgcggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaatccgcgcacgacactgaacatacgaatttaaggaataaagataatggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgatgtaagtttaaacgcggccgcaatttgaacgccagcacatggactctcgagtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggtgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctggacaatgtctccatacccgtttttttgggcgacctcgtcggaggttgtatgtccggtgttccgtgacgtcatcgggcattcatcattcatagaatgtgttacggaggaaacaagtaatggcacttagcaccgcaaccaaggccgcgacggacgcgctggctgccaatcgggcacccaccagcgtgaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcaccaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgaccggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattgaaacgggtgtcgggctccgtcacagggcggggcccgacgcacgatcgttcgatcaacctcaaaccaaaaaggaacatcgatatgagcatgttaggagaaagacgccgcggtctgaccgatccggaaatggcggccgtcattttgaaggcgcttcctgaagctccgctggacggcaacaacaagatgggttatttcgtcaccccccgctggaaacgcttgacggaatatgaagccctgaccgtttatgcgcagcccaacgccgactggatcgccggcggcctggactggggcgactggacccagaaattccacggcggccgcccttcctggggcaacgagaccacggagctgcgcaccgtcgactggttcaagcaccgtgacccgctccgccgttggcatgcgccgtacgtcaaggacaaggccgaggaatggcgctacaccgaccgcttcctgcagggttactccgccgacggtcagatccgggcgatgaacccgacctggcgggacgagttcatcaaccggtattggggcgccttcctgttcaacgaatacggattgttcaacgctcattcgcagggcgcccgggaggcgctgtcggacgtaacccgcgtcagcctggctttctggggcttcgacaagatcgacatcgcccagatgatccaactcgaacggggtttcctcgccaagatcgtacccggtttcgacgagtccacagcggtgccgaaggccgaatggacgaacggggaggtctacaagagcgcccgtctggccgtggaagggctgtggcaggaggtgttcgactggaacgagagcgctttctcggtgcacgccgtctatgacgcgctgttcggtcagttcgtccgccgcgagttctttcagcggctggctccccgcttcggcgacaatctgacgccattcttcatcaaccaggcccagacatacttccagatcgccaagcagggcgtacaggatctgtattacaactgtctgggtgacgatccggagttcagcgattacaaccgtaccgtgatgcgcaactggaccggcaagtggctggagcccacgatcgccgctctgcgcgacttcatggggctgtttgcgaagctgccggcgggcaccactgacaaggaagaaatcaccgcgtccctgtaccgggtggtcgacgactggatcgaggactacgccagcaggatcgacttcaaggcggaccgcgatcagatcgttaaagcggttctggcaggattgaaataatagaggaactattacgatgagcgtaaacagcaacgcatacgacgccggcatcatgggcctgaaaggcaaggacttcgccgatcagttctttgccgacgaaaaccaagtggtccatgaaagcgacacggtcgttctggtcctcaagaagtcggacgagatcaatacctttatcgaggagatccttctgacggactacaagaagaacgtcaatccgacggtaaacgtggaagaccgcgcgggttactggtggatcaaggccaacggcaagatcgaggtcgattgcgacgagatttccgagctgttggggcggcagttcaacgtctacgacttcctcgtcgacgtttcctccaccatcggccgggcctataccctgggcaacaagttcaccattaccagtgagctgatgggcctggaccgcaagctcgaagactatcacgcttaaggagaatgacatggcgaaactgggtatacacagcaacgacacccgcgacgcctgggtgaacaagatcgcgcagctcaacaccctggaaaaagcggccgagatgctgaagcagttccggatggaccacaccacgccgttccgcaacagctacgaactggacaacgactacctctggatcgaggccaagctcgaagagaaggtcgccgtcctcaaggcacgcgccttcaacgaggtggacttccgtcataagaccgctttcggcgaggatgccaagtccgttctggacggcaccgtcgcgaagatgaacgcggccaaggacaagtgggaggcggagaagatccatatcggtttccgccaggcctacaagccgccgatcatgccggtgaactatttcctggacggcgagcgtcagttggggacccggctgatggaactgcgcaacctcaactactacgacacgccgctggaagaactgcgcaaacagcgcggtgtgcgggtggtgcatctgcagtcgccgcactgaagggaggaagtctcgccctggacgcgacggcatcgccgtgaagtccagggggcagggatgccgttccgggccggcaggctggcccggaatctctggttttcagggggcgtgccggtccacggctcccccctccatctttcgtaaggaaatcaccatggtcgaatcggcatttcagccattttcgggcgacgcagacgaatggttcgaggaaccacggccccaggccggtttcttcccttccgcggactggcatctgctcaaacgggacgagacctacgcagcctatgccaaggatctcgatttcatgtggcggtgggtcatcgtccgggaagaaaggatcgtccaggagggttgctcgatcagcctggagtcgtcgatccgcgccgtgacgcacgtactgaattattttggtatgaccgaacaacgcgccccggcagaggaccggaccggcggagttcaacattgaacaggtaagtttatgcagcgagttcacactatcacggcggtgacggaggatggcgaatcgctccgcttcgaatgccgttcggacgaggacgtcatcaccgccgccctgcgccagaacatctttctgatgtcgtcctgccgggagggcggctgtgcgacctgcaaggccttgtgcagcgaaggggactacgacctcaagggctgcagcgttcaggcgctgccgccggaagaggaggaggaagggttggtgttgttgtgccggacctacccgaagaccgacctggaaatcgaactgccctatacccattgccgcatcagttttggtgaggtcggcagtttcgaggcggaggtcgtcggcctcaactgggtttcgagcaacaccgtccagtttcttttgcagaagcggcccgacgagtgcggcaaccgtggcgtgaaattcgaacccggtcagttcatggacctgaccatccccggcaccgatgtctcccgctcctactcgccggcgaaccttcctaatcccgaaggccgcctggagttcctgatccgcgtgttaccggagggacggttttcggactacctgcgcaatgacgcgcgtgtcggacaggtcctctcggtcaaagggccactgggcgtgttcggtctcaaggagcggggcatggcgccgcgctatttcgtggccggcggcaccgggttggcgccggtggtctcgatggtgcggcagatgcaggagtggaccgcgccgaacgagacccgcatctatttcggtgtgaacaccgagccggaattgttctacatcgacgagctcaaatccctggaacgatcgatgcgcaatctcaccgtgaaggcctgtgtctggcacccgagcggggactgggaaggcgagcagggctcgcccatcgatgcgttgcgggaagacctggagtcctccgacgccaacccggacatttatttgtgcggtccgccgggcatgatcgatgccgcctgcgagctggtacgcagccgcggtatccccggcgaacaggtcttcttcgaaaaattcctgccgtccggggcggcctgaaccggggaagtaccgtgaccaccgagcagttcccgccccaattcctgcgtgaaatgatcgagcagctggacgccagcatccaggagctcgcacgcaaggaaaagggacttgcggcatccctgggcacgggccgggtcgccgagctcaaggaatactgggaccacgttgttacaaccaattaaccaattctgactatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgc

SEQ ID NO: 2 - DNA, 13237 bp, Methylococcus capsulatus
  misc_feature 1..13237: note = pNH265
  source 1..13237: mol_type = genomic DNA
ttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggctggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctcccaatgtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttttgacggctagctcagtcctagggataatgctagcaccagcctcgagggaaaccacgtaagctccggcgtttaaacacccataacagatacggactttctcaaaggagagttatcagtgaaaatccgcccgttacatgaccgtgtcatcatcaaacgcttggaagaagagcgtacctcggcgggcgggattgtcattccagatagcgcagctgaaaaaccgatgcgtggtgaaatcctggcagtgggcaatggaaaagtgcttgataatggagaggtacgtgctttacaggtgaaagtgggtgataaagtgctctttgggaaatacgcgggtacggaggttaaagtagatggggaagatgttgttgtcatgcgtgaagatgacattctggctgtgttagaatcttaatccgcgcacgacactgaacatacgaatttaaggaataaagataatggcgaaagaagttgtgtatcgtggtagtgcgcgccagcgtatgatgcagggtattgaaattctcgctcgcgccgctattccaacgctgggggcaaccggcccgagcgtcatgattcaacatcgcgccgatggtctgccacccatttctacacgcgatggcgttaccgtagcgaattctattgttttaaaagaccgtgtcgcgaacctgggtgcccgcctgctgcgcgacgtagccggtacaatgagccgtgaagccggcgacggcacgacgactgcgatcgtattggcccgccacatcgcccgtgagatgtttaaatcgctggccgtgggtgcagatccgatcgcgctgaaacgtggtatcgatcgcgccgttgctcgtgtgtccgaagatattggggcgcgtgcgtggcgtggcgataaagaaagcgtgatcctgggtgtcgctgctgtggcgacgaaaggcgaaccgggcgttggccgtctgctgctggaggctctcgatgcagtgggtgttcacggtgccgtttctatcgaactgggccaacgtcgtgaagatctgctggacgtcgtcgatggctatcgctgggaaaaaggttatttatctccctactttgtcacggaccgtgcccgcgaactcgcggaactggaggatgtctacctgctcatgaccgaccgcgaagtggttgacttcatcgaccttgtacctctgctggaggccgtgacggaagcaggaggctccctgctgattgccgcggatcgtgtgcacgaaaaggccttagcggggctgcttctgaatcacgtgcgcggtgtcttcaaggccgtggccgtaaccgctccgggttttggcgacaaacgcccgaaccgtttacttgacctggccgcgttaaccggcggtcgtgccgtgctcgaagctcaaggcgaccgtctggaccgtgttaccctcgcggatctgggccgtgtgcgccgtgccgtggtgtcggcagatgataccgcgctgcttggcatcccgggcaccgaagctagccgtgcacgcctcgaaggtctgcgtttagaagcagagcagtaccgtgcgctgaaaccagggcagggttctgccaccgggcgcctgcacgaacttgaagaaattgaagcgcgcattgtgggtctgtccggaaagagcgccgtttatcgcgtcggaggtgtgaccgatgtggaaatgaaagagcgcatggttcgcatcgaaaacgcttaccgttcggtggtaagtgcgctggaggaaggcgtgctccctggcggtggtgtcggctttctgggtagtatgccggtgcttgcggaattggaggcccgcgacgcagatgaagctcgcgggattgggattgtacgcagcgccttaacggagcctcttcgtattatcggcgaaaatagtggcttgagcggtgaagccgttgttgccaaagtcatggatcatgccaacccgggatggggttacgaccaggagtctggctctttttgcgacctgcatgcgcgtgggatctgggatgctgctaaagtgttacgtctcgcgttggagaaggcagcctctgttgctgggacctttctgacaaccgaagctgttgttctcgaaattccggatacagatgcgttcgcagggttcagtgcagaatgggctgccgccacgcgcgaagatccgcgcgtatgagtttaaacgcggccgcaatttgaacgcacccataacagatacggactttctcaaaggagagttatcaatgaatattcgtccattgcatgatcgcgtgatcgtcaagcgtaaagaagttgaaactaaatctgctggcggcatcgttctgaccggctctgcagcggctaaatccacccgcggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaatccgcgcacgacactgaacatacgaatttaaggaataaagataatggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgatgtaagtttaaacgcggccgcaatttgaacgccagcacatggactctcgagtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggtgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctggacaatgtctccatacccgtttttttgggcgacctcgtcggaggttgtatgtccggtgttccgtgacgtcatcgggcattcatcattcatagaatgtgttacggaggaaacaagtaatggcacttagcaccgcaaccaaggccgcgacggacgcgctggctgccaatcgggcacccaccagcgtgaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcaccaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgaccggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattgaaacgggtgtcgggctccgtcacagggcggggcccgacgcacgatcgttcgatcaacctcaaaccaaaaaggaacatcgatatgagcatgttaggagaaagacgccgcggtctgaccgatccggaaatggcggccgtcattttgaaggcgcttcctgaagctccgctggacggcaacaacaagatgggttatttcgtcaccccccgctggaaacgcttgacggaatatgaagccctgaccgtttatgcgcagcccaacgccgactggatcgccggcggcctggactggggcgactggacccagaaattccacggcggccgcccttcctggggcaacgagaccacggagctgcgcaccgtcgactggttcaagcaccgtgacccgctccgccgttggcatgcgccgtacgtcaaggacaaggccgaggaatggcgctacaccgaccgcttcctgcagggttactccgccgacggtcagatccgggcgatgaacccgacctggcgggacgagttcatcaaccggtattggggcgccttcctgttcaacgaatacggattgttcaacgctcattcgcagggcgcccgggaggcgctgtcggacgtaacccgcgtcagcctggctttctggggcttcgacaagatcgacatcgcccagatgatccaactcgaacggggtttcctcgccaagatcgtacccggtttcgacgagtccacagcggtgccgaaggccgaatggacgaacggggaggtctacaagagcgcccgtctggccgtggaagggctgtggcaggaggtgttcgactggaacgagagcgctttctcggtgcacgccgtctatgacgcgctgttcggtcagttcgtccgccgcgagttctttcagcggctggctccccgcttcggcgacaatctgacgccattcttcatcaaccaggcccagacatacttccagatcgccaagcagggcgtacaggatctgtattacaactgtctgggtgacgatccggagttcagcgattacaaccgtaccgtgatgcgcaactggaccggcaagtggctggagcccacgatcgccgctctgcgcgacttcatggggctgtttgcgaagctgccggcgggcaccactgacaaggaagaaatcaccgcgtccctgtaccgggtggtcgacgactggatcgaggactacgccagcaggatcgacttcaaggcggaccgcgatcagatcgttaaagcggttctggcaggattgaaataatagaggaactattacgatgagcgtaaacagcaacgcatacgacgccggcatcatgggcctgaaaggcaaggacttcgccgatcagttctttgccgacgaaaaccaagtggtccatgaaagcgacacggtcgttctggtcctcaagaagtcggacgagatcaatacctttatcgaggagatccttctgacggactacaagaagaacgtcaatccgacggtaaacgtggaagaccgcgcgggttactggtggatcaaggccaacggcaagatcgaggtcgattgcgacgagatttccgagctgttggggcggcagttcaacgtctacgacttcctcgtcgacgtttcctccaccatcggccgggcctataccctgggcaacaagttcaccattaccagtgagctgatgggcctggaccgcaagctcgaagactatcacgcttaaggagaatgacatggcgaaactgggtatacacagcaacgacacccgcgacgcctgggtgaacaagatcgcgcagctcaacaccctggaaaaagcggccgagatgctgaagcagttccggatggaccacaccacgccgttccgcaacagctacgaactggacaacgactacctctggatcgaggccaagctcgaagagaaggtcgccgtcctcaaggcacgcgccttcaacgaggtggacttccgtcataagaccgctttcggcgaggatgccaagtccgttctggacggcaccgtcgcgaagatgaacgcggccaaggacaagtgggaggcggagaagatccatatcggtttccgccaggcctacaagccgccgatcatgccggtgaactatttcctggacggcgagcgtcagttggggacccggctgatggaactgcgcaacctcaactactacgacacgccgctggaagaactgcgcaaacagcgcggtgtgcgggtggtgcatctgcagtcgccgcactgaagggaggaagtctcgccctggacgcgacggcatcgccgtgaagtccagggggcagggatgccgttccgggccggcaggctggcccggaatctctggttttcagggggcgtgccggtccacggctcccccctccatctttcgtaaggaaatcaccatggtcgaatcggcatttcagccattttcgggcgacgcagacgaatggttcgaggaaccacggccccaggccggtttcttcccttccgcggactggcatctgctcaaacgggacgagacctacgcagcctatgccaaggatctcgatttcatgtggcggtgggtcatcgtccgggaagaaaggatcgtccaggagggttgctcgatcagcctggagtcgtcgatccgcgccgtgacgcacgtactgaattattttggtatgaccgaacaacgcgccccggcagaggaccggaccggcggagttcaacattgaacaggtaagtttatgcagcgagttcacactatcacggcggtgacggaggatggcgaatcgctccgcttcgaatgccgttcggacgaggacgtcatcaccgccgccctgcgccagaacatctttctgatgtcgtcctgccgggagggcggctgtgcgacctgcaaggccttgtgcagcgaaggggactacgacctcaagggctgcagcgttcaggcgctgccgccggaagaggaggaggaagggttggtgttgttgtgccggacctacccgaagaccgacctggaaatcgaactgccctatacccattgccgcatcagttttggtgaggtcggcagtttcgaggcggaggtcgtcggcctcaactgggtttcgagcaacaccgtccagtttcttttgcagaagcggcccgacgagtgcggcaaccgtggcgtgaaattcgaacccggtcagttcatggacctgaccatccccggcaccgatgtctcccgctcctactcgccggcgaaccttcctaatcccgaaggccgcctggagttcctgatccgcgtgttaccggagggacggttttcggactacctgcgcaatgacgcgcgtgtcggacaggtcctctcggtcaaagggccactgggcgtgttcggtctcaaggagcggggcatggcgccgcgctatttcgtggccggcggcaccgggttggcgccggtggtctcgatggtgcggcagatgcaggagtggaccgcgccgaacgagacccgcatctatttcggtgtgaacaccgagccggaattgttctacatcgacgagctcaaatccctggaacgatcgatgcgcaatctcaccgtgaaggcctgtgtctggcacccgagcggggactgggaaggcgagcagggctcgcccatcgatgcgttgcgggaagacctggagtcctccgacgccaacccggacatttatttgtgcggtccgccgggcatgatcgatgccgcctgcgagctggtacgcagccgcggtatccccggcgaacaggtcttcttcgaaaaattcctgccgtccggggcggcctgaaccggggaagtaccgtgaccaccgagcagttcccgccccaattcctgcgtgaaatgatcgagcagctggacgccagcatccaggagctcgcacgcaaggaaaagggacttgcggcatccctgggcacgggccgggtcgccgagctcaaggaatactgggaccacgttgttacaaccaattaaccaattctgactatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcg

SEQ ID NO: 3 - protein, 527 aa, Methylococcus capsulatus
  REGION 1..527: note = misc_feature - mmoX
MALSTATKAATDALAANRAPTSVNAQEVHRWLQSFNWDFKNNRTKYATKYKMANETKEQFKLIAKEYARMEAVKDERQFGSLQDALTRLNAGVRVHPKWNETMKVVSNFLEVGEYNAIAATGMLWDSAQAAEQKNGYLAQVLDEIRHTHQCAYVNYYFAKNGQDPAGHNDARRTRTIGPLWKGMKRVFSDGFISGDAVECSLNLQLVGEACFTNPLIVAVTEWAAANGDEITPTVFLSIETDELRHMANGYQTVVSIANDPASAKYLNTDLNNAFWTQQKYFTPVLGMLFEYGSKFKVEPWVKTWNRWVYEDWGGIWIGRLGKYGVESPRSLKDAKQDAYWAHHDLYLLAYALWPTGFFRLALPDQEEMEWFEANYPGWYDHYGKIYEEWRARGCEDPSSGFIPLMWFIENNHPIYIDRVSQVPFCPSLAKGASTLRVHEYNGQMHTFSDQWGERMWLAEPERYECQNIFEQYEGRELSEVIAELHGLRSDGKTLIAQPHVRGDKLWTLDDIKRLNCVFKNPVKAFN

SEQ ID NO: 4 - protein, 389 aa, Methylococcus capsulatus
  REGION 1..389: note = misc_feature - mmoY
MSMLGERRRGLTDPEMAAVILKALPEAPLDGNNKMGYFVTPRWKRLTEYEALTVYAQPNADWIAGGLDWGDWTQKFHGGRPSWGNETTELRTVDWFKHRDPLRRWHAPYVKDKAEEWRYTDRFLQGYSADGQIRAMNPTWRDEFINRYWGAFLFNEYGLFNAHSQGAREALSDVTRVSLAFWGFDKIDIAQMIQLERGFLAKIVPGFDESTAVPKAEWTNGEVYKSARLAVEGLWQEVFDWNESAFSVHAVYDALFGQFVRREFFQRLAPRFGDNLTPFFINQAQTYFQIAKQGVQDLYYNCLGDDPEFSDYNRTVMRNWTGKWLEPTIAALRDFMGLFAKLPAGTTDKEEITASLYRVVDDWIEDYASRIDFKADRDQIVKAVLAGLK

SEQ ID NO: 5 - protein, 170 aa, Methylococcus capsulatus
  REGION 1..170: note = misc_feature - mmoZ
MAKLGIHSNDTRDAWVNKIAQLNTLEKAAEMLKQFRMDHTTPFRNSYELDNDYLWIEAKLEEKVAVLKARAFNEVDFRHKTAFGEDAKSVLDGTVAKMNAAKDKWEAEKIHIGFRQAYKPPIMPVNYFLDGERQLGTRLMELRNLNYYDTPLEELRKQRGVRVVHLQSPH

SEQ ID NO: 6 - protein, 141 aa, Methylococcus capsulatus
  REGION 1..141: note = misc_feature - mmoB
MSVNSNAYDAGIMGLKGKDFADQFFADENQVVHESDTVVLVLKKSDEINTFIEEILLTDYKKNVNPTVNVEDRAGYWWIKANGKIEVDCDEISELLGRQFNVYDFLVDVSSTIGRAYTLGNKFTITSELMGLDRKLEDYHA

SEQ ID NO: 7 - protein, 348 aa, Methylococcus capsulatus
  REGION 1..348: note = misc_feature - mmoC
MQRVHTITAVTEDGESLRFECRSDEDVITAALRQNIFLMSSCREGGCATCKALCSEGDYDLKGCSVQALPPEEEEEGLVLLCRTYPKTDLEIELPYTHCRISFGEVGSFEAEVVGLNWVSSNTVQFLLQKRPDECGNRGVKFEPGQFMDLTIPGTDVSRSYSPANLPNPEGRLEFLIRVLPEGRFSDYLRNDARVGQVLSVKGPLGVFGLKERGMAPRYFVAGGTGLAPVVSMVRQMQEWTAPNETRIYFGVNTEPELFYIDELKSLERSMRNLTVKACVWHPSGDWEGEQGSPIDALREDLESSDANPDIYLCGPPGMIDAACELVRSRGIPGEQVFFEKFLPSGAA

SEQ ID NO: 8 - protein, 103 aa, Methylococcus capsulatus
  REGION 1..103: note = misc_feature - mmoD
MVESAFQPFSGDADEWFEEPRPQAGFFPSADWHLLKRDETYAAYAKDLDFMWRWVIVREERIVQEGCSISLESSIRAVTHVLNYFGMTEQRAPAEDRTGGVQH

SEQ ID NO: 9 - protein, 559 aa, Methylococcus capsulatus
  REGION 1..559: note = misc_feature - groEL-2
MAKEVVYRGSARQRMMQGIEILARAAIPTLGATGPSVMIQHRADGLPPISTRDGVTVANSIVLKDRVANLGARLLRDVAGTMSREAGDGTTTAIVLARHIAREMFKSLAVGADPIALKRGIDRAVARVSEDIGARAWRGDKESVILGVAAVATKGEPGVGRLLLEALDAVGVHGAVSIELGQRREDLLDVVDGYRWEKGYLSPYFVTDRARELAELEDVYLLMTDREVVDFIDLVPLLEAVTEAGGSLLIAADRVHEKALAGLLLNHVRGVFKAVAVTAPGFGDKRPNRLLDLAALTGGRAVLEAQGDRLDRVTLADLGRVRRAVVSADDTALLGIPGTEASRARLEGLRLEAEQYRALKPGQGSATGRLHELEEIEARIVGLSGKSAVYRVGGVTDVEMKERMVRIENAYRSVVSALEEGVLPGGGVGFLGSMPVLAELEARDADEARGIGIVRSALTEPLRIIGENSGLSGEAVVAKVMDHANPGWGYDQESGSFCDLHARGIWDAAKVLRLALEKAASVAGTFLTTEAVVLEIPDTDAFAGFSAEWAAATREDPRV

(19) United States
(12) Patent Application Publication
(10) Pub. No.: US 2024/0309339 A1
(43) Pub. Date: Sep. 19, 2024

(54) METHANE MONOOXYGENASE ENZYMES

(71) Applicant: Industrial Microbes, Inc., Alameda, CA (US)

(72) Inventors: Elizabeth Jane Clarke, San Francisco, CA (US); Derek Lorin Greenfield, Kinsignton, CA (US); Noah Charles Helman, El Cerrito, CA (US); Stephanie Rhianon Jones, Berkeley, CA (US); Baolong Zhu, Johnston, IA (US)

(21) Appl. No.: 18/393,317

(22) Filed: Dec. 21, 2023

Related U.S. Application Data
(62) Division of application No. 17/461,102, filed on Aug. 30, 2021, now Pat. No. 11,851,686.
Application No. 17/461,102 is a continuation of application No. 16/629,018, filed on Jan. 6, 2020, now abandoned.
(60) Provisional application No. 62/529,648, filed on Jul. 7, 2017.
(60) Provisional application No. 62/542,838, filed on Aug. 9, 2017.
(60) Provisional application No. 62/566,733, filed on Oct. 2, 2017.

(51) Int. Cl. C12N 9/02; C12N 15/70; C12N 15/75; C12N 15/77

(52) U.S. Cl. CPC C12N 9/0073; C12N 15/70; C12N 15/75; C12N 15/77; C12Y 114/13025

(57) ABSTRACT

Improved soluble methane monooxygenases and soluble methane monooxygenase systems are provided.

### CROSS-REFERENCE TO RELATED APPLICATIONS

This application claims priority to U.S. provisional application No. 62/529,648, filed Jul. 7, 2017; U.S. provisional application No. 62/542,838, filed Aug. 9, 2017; and U.S. provisional application No. 62/566,733, filed Oct. 2, 2017, each of which is incorporated by reference herein in its entirety, including any drawings.

This invention was made with government support under Department of Energy Agreement DE-AR0000432, awarded by the Advanced Research Projects Agency-Energy (ARPA-E). The government has certain rights in the invention.

### SEQUENCE LISTING

The instant application contains a Sequence Listing which has been submitted electronically in XML format and is hereby incorporated by reference in its entirety. Said XML copy, created on May 13, 2024, is named 1107367.00052_SL.xml and is 40,919 bytes in size.

### FIELD

The technology provided herein relates to engineered enzymes applicable for industrial biotechnology. In particular, the technology relates to improved soluble methane monooxygenases and soluble methane monooxygenase systems.

### BACKGROUND

Recent technological advances in synthetic biology have demonstrated the power and versatility of enzymatic pathways in living cells to convert organic molecules into industrial products. The petrochemical processes that currently manufacture these industrial products may be replaced by biotechnological processes that can often provide the same products at a lower cost and with a lower environmental impact. The discovery of new pathways and enzymes that can operate and be engineered in genetically tractable microorganisms will further advance synthetic biology.

Sugar, including simple sugars, starches, carbohydrates, and sugar alcohols, is often used as a raw material for biological fermentations. But, sugar is relatively expensive as a raw material, which severely limits the economic viability of any fermentation process that uses sugar as a raw material. When using biological fermentation to produce chemicals, companies often must limit themselves to the production of only select chemicals due to the high cost of sugar.

Other raw materials may be used to produce chemicals. Short alkanes, such as methane and ethane, are significantly less expensive as raw materials than sugar. Given the enormous supply of natural gas and the emergence of renewable methane-production technologies, short alkanes are expected to remain inexpensive for decades to come. Industrial products made by engineered microorganisms from short alkanes would be less expensive to manufacture than those made by sugar and should remain so for decades.

Any biological system capable of converting short alkanes into industrial products must include an enzyme that can activate the alkane. Naturally occurring bacteria that can activate methane use dioxygen to convert methane to methanol. An example of an enzyme capable of activating methane belongs to the class of enzymes known as soluble diiron monooxygenases.

There has been some difficulty expressing soluble diiron monooxygenases in industrially relevant hosts. There are many desirable factors involved in optimizing an enzyme for industrial use, such as high turnover rate, specificity for the desired substrate (and against any unintended substrates), solubility, high substrate binding affinity, reduced product inhibition, cofactor preference, and others. Successful functional expression of soluble diiron monooxygenases is critical for being able to use inexpensive raw materials in industrially relevant hosts.

What is needed is a soluble diiron monooxygenase with improved function that can be expressed in an industrially relevant host.

### SUMMARY

The invention provided herein is drawn to improved soluble diiron monooxygenase mutants that can be functionally expressed in industrially relevant hosts.

Some embodiments provide at least one mutant soluble diiron monooxygenase system comprising at least one mutant subunit, wherein the at least one mutant subunit comprises one or more mutations, such that the activity of the mutant soluble diiron monooxygenase system is greater than the activity of a wild-type soluble diiron monooxygenase system.

In some embodiments, the at least one mutant soluble diiron monooxygenase system comprises a mutant hydroxylase alpha subunit, wherein the mutant hydroxylase alpha subunit comprises one or more mutations at one or more residue position(s): 3, 14, 25, 26, 32, 33, 35, 42, 44, 53, 54, 55, 56, 57, 61, 130, 135, 154, 162, 163, 168, 216, 251, 262, 263, 264, 277, 316, 322, 330, 347, 356, 374, 375, 381, 387, 393, 399, 405, 419, 420, 422, 431, 432, 433, 434, 434, 444, 453, 456, 459, 469, 470, 475, 480, 485, 493, 504, 505, 510, 513, 515, 525, and/or 526, wherein the residue position(s) are numbered with respect to SEQ ID NO: 3. In some embodiments, the one or more mutations comprises substitutions at one or more of residue position(s): L3P, L14P, A25S, Q26G, L32P, Q33K, Q33R, F35L, N42R, T44A, T44S, A53G, N54K, N54L, E55A, E55S, T56A, T56G, K57G, K57S, K57T, K61A, K61R, A130C, A130G, A130H, A130T, N135S, V154L, G162C, Q163F, H168G, L216M, Y251M, Y251W, A262E, S263Q, A264Q, T277G, 1316S, G322N, R330T, Y347G, T356C, T356G, T356M, A374E, N375H, D381R, D381S, Y387I, Y387L, R393C, R399D, L405G, R419L, R419S, V420C, Q422E, K431E, G432A, A433G, A433Q, A433T, S434G, S434N, Q444E, G453V, M456V, A459E, 1469V, F470I, G475*, E480G, L485E, K493E, K493V, D504E, K505E, D510E, K513R, K513T, L515G, A525Q, F526C, F526G, F526L, and/or F526S, wherein the residue position(s) are numbered with respect to SEQ ID NO: 3. In some embodiments, the one or more mutations consist of substitutions at residue position(s):
a. L3P;
b. L3P, L14P;
c. L3P, L14P, L32P, F35L, and R330T;
d. L3P, L14P, and S434N;
e. A25S;
f. Q26G;
g. L32P and A262E;
h. Q33K;
i. Q33R;
j. N42R and T44A;
k. T44S;
l. A53G;
m. N54K;
n. N54L;
o. E55A;
p. E55S and N375H;
q. T56A;
r. T56G;
s. K57G;
t. K57S;
u. K57T;
v. K61A;
w. K61R;
x. A130C;
y. A130C and R419L;
z. A130H;
aa. A130T;
ab. N135S;
ac. V154L;
ad. V154L and S263Q;
ae. G162C;
af. Q163F;
ag. H168G;
ah. L216M;
ai. Y251M;
aj. Y251W;
ak. A264Q;
al. T277G;
am. 1316S;
an. G322N;
ao. Y347G;
ap. T356C;
aq. T356G;
ar. T356M;
as. A374E;
at. D381R;
au. D381S;
av. Y387I;
aw. Y387L;
ax. R393C;
ay. S399D;
az. L405G;
ba. R419S;
bb. V420C;
bc. Q422E, K431E, E480G, and D504E;
bd. K431E;
be. G432A;
bf. A433G and A525Q;
bg. A433Q;
bh. A433T;
bi. S434G and K505E;
bj. S434N;
bk Q444E;
bl. G453V;
bm. M456V;
bn. A459E and K513T;
bo. I469V;
bp. F470I and G475*;
bq. LA85E;
br. K493E;
bs. K493V;
bt. K505E;
bu. D510E;
bv. K513R;
bw. K513T;
bx. L515G;
by. F526C;
bz. F526G;
ca. F526L; or
cb. F5265,
   wherein the residue position(s) are numbered with respect to SEQ ID NO: 3.

In some embodiments, the at least one mutant soluble diiron monooxygenase system comprises a mutant accessory protein B, wherein the mutant accessory protein B comprises one or more mutations at one or more residue position(s): 3, 15, 16, 38, 58, 69, 81, 101, 102, 103, 106, 110, 112, 116, 118, 119, 120, 124, 126, 130, 131, 134, 136, 138, and/or 139, wherein the residue position(s) are numbered with respect to SEQ ID NO: 6. In some embodiments, the one or more mutations comprises substitutions at one or more residue position(s): V3G, L15M, K16G, V38G, T58E, N69K, A81G, N101G, V102A, V102L, Y103H, L106V, S110A, T112G, T112V, A116S, A116T, T118E, T118G, L119E, G120A, G120E, T124C, T124G, T126S, M130D, M130V, G131S, R134A, R134G, R134W, L136A, D138S, Y139G, Y139S, and/or Y139V, wherein the residue position(s) are numbered with respect to SEQ ID NO: 6. In some embodiments, the one or more mutations consists of a substitution at residue position(s):
a. V3G;
b. L15M;
c. K16G;
d. V38G;
e. T58E and T118E;
f. N69K;
g. A81G;
h. N101G;
i. V102A;
j. V102L;
k. Y103H;
l. L106V;
m. S110A;
n. T112G;
o. T112V;
p. A116S;
q. A116T;
r. T118G;
s. L119E;
t. G120A;
u. G120E;
v. T124C;
w. T124G;
x. T126S;
y. M130D;
z. M130V;
aa. G131S;
ab. R134A;
ac. R134G;
ad. R134W;
ae. L136A;
af. D138S;
ag. Y139G;
ah. Y139S; or
ai. Y139V,
   wherein the residue position(s) are numbered with respect to SEQ ID NO: 6.

In some embodiments, the at least one mutant soluble diiron monooxygenase system comprises a mutant reductase subunit C, wherein the mutant reductase subunit C further comprises one or more mutations at one or more residue position(s): 17, 28, 38, 44, 45, 49, 58, 62, 64, 69, 74, 87, 90, 97, 98, 100, 110, 111, 116, 119, 125, 133, 135, 138, 143, 144, 152, 153, 155, 157, 174, 180, 184, 191, 196, 204, 208, 209, 211, 213, 216, 222, 235, 236, 238, 240, 242, 243, 244, 245, 246, 279, 281, 286, 288, 308, 324, 329, and/or 330, wherein the residue position(s) are numbered with respect to SEQ ID NO: 7. In some embodiments, the one or more mutations comprises substitutions at one or more residue position(s): L17R, L17Q, I28G, L38G, E44G, E44R, G45R, T49L, D58K, K62L, C64A, C64Q, L69A, E74R, K87L, L90Q, T97V, H98G, R100G, E110R, A111V, L116V, V119C, Q125L, D133V, C135T, R138Q, E143K, P144C, I152S, P153H, T155A, V157R, E174A, L180S, R184G, N191D, G196V, P204S, F208Q, G209W, K211V, R213G, A216V, A222G, R235G, Q236M, Q238G, W240E, A242V, P243G, N244G, E245D, E245M, T246M, C279S, W281R, D286T, E288V, N308V, C324G, S329G, and/or R330W, wherein the residue position(s) are numbered with respect to SEQ ID NO: 7. In some embodiments, the one or more mutations consist of a substitution at residue position(s):
a. L17R and W240E;
b. L17Q;
c. I28G;
d. L38G;
e. E44R;
f. E44G;
h. G45R and S329G;
i. T49L;
j. D58K;
k. K62L;
l. C64Q;
m. C64A and R330W;
n. L69A;
o. E74R;
p. K87L and Q236M;
q. L90Q and A216V;
r. T97V;
s. H98G;
t. R100G and E288V;
u. E110R and N308V;
v. A111V;
w. L116V and K211V;
x. V119C;
y. Q125L;
z. D133V and D286T;
aa. C135T;
ab. R138Q and F208Q;
ac. E143K and P144C;
ad. I152S;
ae. P153H;
af. T155A and C279S;
ag. V157R;
ah. L180S;
ai. R184G;
aj. N191D;
ak. N196V;
al. P204S;
am. G209W;
an. R213G and W281R;
ao. A222G;
ap. R235G;
aq. Q238G;
ar. A242V;
as. P243G and E245D;
at. N244G;
au. E245M;
av. T246M; or
aw. C324G,
   wherein the residue position(s) are numbered with respect to SEQ ID NO: 7.

In some embodiments, the at least one mutant soluble diiron monooxygenase system comprises a mutant hydroxylase beta subunit, wherein the mutant hydroxylase beta subunit comprises one or more mutations at one or more residue position(s): 32, 36, 46, 47, 58, 64, 87, 93, 109, 133, 143, 146, 151, 155, 157, 160, 162, 202, 167, 182, 183, 184, 186, 201, 202, 203, 204, 206, 208, 209, 211, 212, 213, 214, 216, 217, 220, 222, 224, 225, 226, 230, 233, 236, 239, 242, 244, 250, 256, 260, 266, 269, 270, 272, 273, 276, 281, 291, 303, 304, 305, 306, 308, 310, 311, 314, 318, 322, 325, 328, 329, 330, 333, 335, 336, 356, 364, and/or 372, wherein the residue position(s) are numbered with respect to SEQ ID NO: 4. In some embodiments, the one or more mutations comprises substitutions at one or more residue position(s): N32V, G36L, L46V, T47H, T47V, P58N, A64G, T87V, V93G, Y109A, Y109E, Y109F, Y109V, 1133S, E143T, N146R, A151G, N155G, Y157W, F160K, F160M, F160T, F160V, A162T, 202, A167Q, W182M, G183A, G183S, F184S, K186N, A201G, K202A, K202Q, K202R, K202S, I203V, V204S, G206E, D208A, E209R, T211I, A212G, V213G, V213W, P214V, A216G, E217L, N220V, E222R, Y224M, K225G, S226G, A230G, G233L, G233R, G233W, Q236G, Q236V, Q236W, F239L, N242G, N242P, N242V, S244G, A250C, F256W, V260C, Q266V, A269G, P270R, F272K, G273T, G273V, L276V, I281G, A291T, L303G, G304A, D305A, D305G, D305L, D305R, D305W, D306E, D306Q, D306R, E308G, S310R, S310W, D311A, D311G, R314G, R314H, R314Q, R318G, R318I, G322R, L325G, T328G, 1329W, A330R, R333V, F335G, M336G, L356G, I364G, and/or D372W, wherein the residue position(s) are numbered with respect to SEQ ID NO: 4. In some embodiments, the one or more mutations consist of a substitution at residue position(s):
a. N32V;
b. G36L;
c. LA6V;
d. T47H and A151G;
e. T47V;
f. P58N;
g. A64G;
h. T87V;
i. V93G;
j. Y109A;
k. Y109E;
l. Y109F;
m. Y109V;
n. I133S;
o. E143T;
p. N146R;
q. N155G;
r. Y157W;
s. F160K;
t. F160M;
u. F160T;
v. F160V;
w. A162T and K202Q;
x. A167Q;
y. W182M;
z. G183A;
aa. G183S;
ab. F184S;
ac. K186N;
ad. A201G and G233R;
ae. K202A;
af. K202R and A203G;
ag. K202S;
ah. I203V and G273T;
ai. V204S;
aj. G206E;
ak. D208A and R318I;
al. E209R;
am. T211I;
an. A212G and A330R;
ao. A212G;
ap. V213G;
aq. V213 W;
ar. P214V;
as. A216G and G273V;
at. E217L;
au. N220V;
av. E222R and D305L;
aw. Y224M;
ax. K225G and R333V;
ay. S226G;
az. G233L;
ba. G233W;
bb. Q236G;
bc. Q236V;
bd. Q236W;
be. F239L;
bf. N242G;
bg. N242P and A291T;
bh. N242V;
bi. S244G and 1281G;
bj. A250C and L325G;
bk. F256W;
bl. V260C;
bm. Q266V;
bn. A269G;
bo. P270R and D306Q;
bp. F272K;
bq. L276V;
bs. L303G;
bt. G304A;
bu. D305A;
bv. D305G;
bw. D305R;
bx. D305W;
by. D306E;
bz. D306R;
ca. E308G and R314H;
cb. S310R;
cc. S310W;
cd. D311A;
ce. D311G;
cf. R314G;
cg. R314Q,
ch. R318G;
ci. G322R;
cj. T328G;
ck. I329W;
cl. F335G;
cm. M336G;
cn. L356G;
co. I364G; or
cp. D372W,
   wherein the residue position(s) are numbered with respect to SEQ ID NO: 4.

In some embodiments, the at least one mutant soluble diiron monooxygenase system comprises a mutant hydroxylase gamma subunit, wherein the mutant hydroxylase gamma subunit comprises one or more mutations at one or more residue position(s): 10, 17, 18, 20, 21, 23, 24, 27, 29, 31, 33, 34, 41, 43, 45, 46, 47, 50, 51, 52, 53, 56, 63, 66, 67, 70, 79, 80, 103, 112, 116, 117, 127, 129, 136, 144, 147, 153, 154, 156, 165, and/or 166, wherein the residue position(s) are numbered with respect to SEQ ID NO: 5. In some embodiments, the one or more mutations comprises substitutions at one or more residue position(s): D1OR, D10V, N17F, N17W, K18A, K18E, A20G, Q21H, N23A, N23D, N23E, N23G, N23L, N23V, T24E, T24V, K27G, K27Q, K27R, A29G, M31W, K33V, Q34A, Q34K, T41G, F43K, F43S, F43V, N45G, S46G, S461, Y47S, Y47T, D50G, N51G, N51V, D52G, Y53C, 156L, K63G, K63T, V66D, L67E, R70E, R70G, R7OV, H79E, K80M, D103G, 1112V, Q116S, 1117L, Y127F, L129G, G136Q, G136S, N144D, N144G, N144V, Y147D, E153G, E154G, R156A, H165G, L166E, and/or L166G, wherein the residue position(s) are numbered with respect to SEQ ID NO: 5. In some embodiments, the one or more mutations consist of a substitution at residue position(s):
a. D10R;
b. D10V;
c. N17F;
d. N17W;
e. K18A;
f. K18E;
g. A20G;
h. Q21H and I112V;
i. N23A;
j. N23D;
k. N23E;
l. N23G;
m. N23L;
n. N23L and A117L;
o). N23V;
p. T24E;
q. T24V and F43;
r. K27G;
s. K27Q;
t. K27R;
u. A29G;
v. A29G and E154G;
w. M31W;
x. K33V;
y. Q34A;
z. Q34K;
aa. T41G;
ab. F43S;
ac. F43V;
ad. N45G;
ae. S46G;
af. S461;
ag. Y47S;
ah. Y47T;
ai. D50G;
aj. N51G;
ak. N51V;
al. D52G;
am. Y53C;
an. I56L;
ao. K63G;
ap. K63T;
aq. V66D;
ar. L67E;
as. R70E;
at. R70G;
au. R70V;
av. H79E;
aw. K80M;
ax. D103G and L166E;
ay. Q116S;
az. Y127F;
ba. L129G;
bb. G136Q;
bc. G136S;
bd. N144D;
be. N144G;
bf. N144V;
bg. Y147D;
bh. E153G;
bi. E154G;
bj. R156A;
bk. H165G; or
bl. L166G,
   wherein the residue position(s) are numbered with respect to SEQ ID NO: 5.

In some embodiments, the at least one mutant soluble diiron monooxygenase system comprises a mutant subunit D (mmoD), wherein the mutant subunit D further comprises one or more mutations at one or more residue position(s): 4, 5, 7, 8, 9, 10, 13, 14, 17, 18, 20, 21, 22, 24, 27, 40, 60, 74, 79, 80, 89, 90, 92, 93, and/or 96, wherein the residue position(s) are numbered with respect to SEQ ID NO: 8. In some embodiments, the one or more mutations comprises substitutions at one or more residue position(s): S4R, A5S, Q7G, P8G, F9G, F9G, S10R, A13E, D14R, F17G, F17G, E18R, P20G, R21L, P22R, A24G, F27V, T40R, E60Q, S74A, T79G, T79S, H80G, E89D, Q90S, A92P, P93A, and/or D96R, wherein the residue position(s) are numbered with respect to SEQ ID NO: 8. In some embodiments, the one or more mutations consist of a substitution at residue position(s):
a. S4R;
b. A5S;
c. Q7G;
d. P8G;
e. F9G;
f. S10R;
g. A13E;
h. D14R;
i. F17G;
j. E18R;
k. P20G;
l. R21L;
m. P22R;
n. A24G;
o. F27V;
p. T40R;
q. E60Q;
r. S74A;
s. T79G;
t. T79S;
u. H80G;
v. E89D;
w. Q90S;
x. A92P;
y. P93A; or
z. D96R,
   wherein the residue position(s) are numbered with respect to SEQ ID NO: 8.

In some embodiments, the at least one mutant soluble diiron monooxygenase system comprises a mutant protein folding chaperone groEL-2, wherein the mutant protein folding chaperone groEL-2 further comprises one or more mutations at one or more residue position(s): 109, 160, 168, 169, 171, 182, 187, 396, 405, 409, 438, 444, 461, 466, 478, 484, 485, 495, 522, 524, and/or 529, wherein the residue position(s) are numbered with respect to SEQ ID NO: 9. In some embodiments, the one or more mutations comprises substitutions at one or more residue position(s): A109E, A109P, G160A, D168V, A169S, G171P, Q182W, L187V, T396L, V405G, V405D, N409G, A438K, D444S, P461V, G466L, A478L, A478R, A484E, A484G, N485T, G495H, V522L, G524T, and/or T529G, wherein the residue position(s) are numbered with respect to SEQ ID NO: 9. In some embodiments, the one or more mutations consists of substitutions at residue position(s):
a. A109E;
b. A109P;
c. G160A;
d. D168V;
e. A169S;
f. G171P;
g. Q182W;
h. L187V;
i. T396L;
j. V405G;
k. V405D;
l. N409G;
m. A438K;
n. D444S;
o. P461V;
p. G466L;
q. A478L;
r. A478R;
s. A484E;
t. A484G;
u. N485T;
v. G495H;
w. V522L;
x. G524T; or
y. T529G,
   wherein the residue position(s) are numbered with respect to SEQ ID NO: 9.

In some embodiments, the at least one mutant soluble diiron monooxygenase system comprises or consists of one or more mutant subunits disclosed herein. In some embodiments, the at least one mutant soluble diiron monooxygenase system comprises or consists of one or more of a mutant hydroxylase alpha subunit, a mutant hydroxylase beta subunit, a mutant hydroxylase gamma subunit, a mutant accessory protein B, a mutant reductase subunit C, a mutant subunit D, and/or a mutant protein folding chaperone groEL-2 as disclosed herein.

A second aspect provides one or more isolated nucleic acids encoding one or more polypeptides or one or more subunits comprising or consisting of the mutant soluble diiron monooxygenase subunits or system disclosed herein. Some embodiments provide one or more vectors comprising or consisting of the one or more isolated nucleic acids. Some embodiments provide one or more host cells comprising or consisting of the one or more vectors. In some embodiments, the one or more host cells comprises a prokaryotic cell. In some embodiments, the prokaryotic cell comprises one or more of Escherichia coli, Corynebacterium glutamicum, and/or Bacillus methanolicus. In some embodiments, the one or more host cells comprises Pichia pastoris and/or Saccharomyces cerevisiae.

In some embodiments, at least one mutant soluble diiron monooxygenase system can be used to produce one or more chemicals. In some embodiments, the at least one mutant soluble diiron monooxygenase system comprises one or more mutant subunits as set forth herein, wherein the one or more mutant subunits is purified in vitro. In some embodiments, the at least one mutant soluble diiron monooxygenase system can be used in vitro to produce one or more chemicals.

### BRIEF DESCRIPTION OF THE DRAWINGS

FIG. 1 sets forth a schematic overview of the soluble methane monooxygenase operon of M capsulatus (Bath).

### DESCRIPTION OF THE EXEMPLARY EMBODIMENTS

The present invention relates to at least one mutant soluble diiron monooxygenase system. The at least one mutant soluble diiron monooxygenase system may be composed of multiple subunits, each subunit comprising or consisting of one or more mutations. One or more mutations in any of the subunits improves activity.

### I. Definitions

As used herein, “activity” or “enzyme activity” refers to moles of substrate converted per unit of time. Activity often depends upon conditions and the conditions are sometimes specified. For example, without limitation, activity can be measured using any of the assays set forth in the examples. In some embodiments, activity is measured as set forth in example 1. In some embodiments, activity is measured with respect to the amount of methanol a particular mutant soluble diiron monooxygenase produces as compared to a wild-type soluble diiron monooxygenase. In some embodiments, the at least one mutant soluble diiron monooxygenase system has an increased activity when compared to a wild-type soluble diiron monooxygenase system.

As used herein, the terms “protein folding chaperone,” “folding chaperone,” and “chaperone” refer to one or more proteins that improve the folding of polypeptide chains into 3-dimensional structures. Protein folding chaperones help their substrates, namely other proteins, become properly folded and often more highly soluble. Since most proteins must be folded in a particular shape to be functional, the expression of protein folding chaperones can assist in the proper assembly of certain enzymes in a cell and thereby can result in an increase in the enzymatic activity of the substrate proteins.

As used herein, “conservative amino acid substitution” refers to a substitution in which an amino acid residue is substituted by another amino acid residue having a side chain with similar chemical properties (e.g., charge or hydrophobicity.) In general, a conservative amino acid substitution should not substantially change the functional properties of a protein. The following six groups each contain amino acids that are often, depending upon context, considered conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

As used herein, “directed evolution” refers to a method used in protein engineering that mimics the process of natural selection to evolve proteins or nucleic acids toward a user-defined goal. Directed evolution comprises or consists of subjecting a gene to a method generating genetic diversity, assaying (screening or selecting) the diversity for a property of interest to identify beneficial, neutral, and/or deleterious mutations, and recombining of a subset of mutations which can then be screened for improved mutants.

As used herein, “enzyme” or “enzymatically” refers to biological catalysts. Enzymes accelerate, or catalyze, chemical reactions. Like all catalysts, enzymes increase the rate of reaction by lowering the activation energy.

As used herein, “eukaryotic” refers to organisms whose cells have a nucleus enclosed within membranes. Eukaryotes belong to the domain Eukaryota or Eukarya. Eukaryotic cells also contain other membrane-bound organelles such as mitochondria and the Golgi apparatus, and in addition, some cells of plants and algae contain chloroplasts.

As used herein, “homology” or “homologous” refers to the degree of biological shared ancestry in the evolutionary history of life. Homology or homologous may also refer to sequence homology, the biological homology between protein or polynucleotide sequences with respect to shared ancestry as determined by the closeness of nucleotide or protein sequences. Homology among proteins or polynucleotides is typically inferred from their sequence similarity. Alignments of multiple sequences are used to indicate which regions of each sequence are homologous. The term “percent homology” often refers to “sequence similarity.” The percentage of identical residues (percent identity) or the percentage of residues conserved with similar physiochemical properties (percent similarity) e.g. leucine and isoleucine, is usually used to quantify homology. Partial homology can occur where a segment of the compared sequences has a shared origin.

Homology also refers to multi-gene homology. Thus, homology can also occur with respect to operons or gene clusters as basic units, instead of single genes. Homology can also occur with respect to a system of proteins instead of a single protein. One skilled in the art would know how to use programs such as MultiGeneBlast, a comprehensive BLAST implementation, to perform homology searches on multigene modules (See, Medema, H., Takano, E., and Breitling R., Detecting Sequence Homology at the Gene Cluster Level with MultiGeneBlast, Mol Biol Evol. (2013) May; 30(5): 1218-1223, which is incorporated by reference herein in its entirety, including any drawings). In a multi-gene homology, one skilled in the art can search for all genomic loci containing a combination of certain genes within the same gene cluster or for only certain genes or proteins of interest performing a specified part or combination of enzymatic steps. As it refers to a multi-subunit protein complex, “percent identity” is intended to mean the maximum value for the percent identity between any pairwise combination of amino acid sequences, calculated between all the subunits in one complex measured against all the subunits in the second complex. The percent identity between two subunits can be calculated using publicly available computational tools, such as BLASTp from NCBI.

As used herein, “isolated,” “isolating,” or “isolate” refers to a process of purification of a nucleic acid from a sample using a combination of physical and chemical methods.

As used herein, “nucleic acid,” “polynucleotide,” or “oligonucleotide” each refer to one or more polymers of nucleic acids and include, but are not limited to, coding regions, which are transcribed or translated into a polypeptide or chaperone, appropriate regulatory or control sequences, controlling sequences, e.g., translational start and stop codons, promoter sequences, ribosome binding sites, polyadenylation signals, transcription factor binding sites, termination sequences, regulatory domains, and enhancers, among others. A polynucleotide need not include all of its relevant or even complete coding regions on a single polymer and the invention provided herein contemplates having complete or partial coding regions on different polymers.

As used herein, “mutant,” “mutant subunit,” or “mutations” refers to an alteration of the nucleotide sequence of a genome of an organism, virus, or extrachromosomal nucleic acid or other genetic elements.

As used herein, “polypeptide” refers to a polymer consisting of a number of amino-acid residues bonded together in a chain, forming part of (or the whole of) a protein molecule.

As used herein, “prokaryotic” refers to a microscopic single-celled organism that has neither a distinct nucleus with a membrane nor other specialized organelles. Prokaryotes include bacteria and cyanobacteria.

As used herein, “soluble diiron monooxygenase” refers to a class of enzymes and enzyme complexes characterized by a catalytic core of two iron atoms and the ability to utilize molecular oxygen to catalyze hydroxylation or epoxidation of hydrocarbon bonds. The enzymes typically require NADH or NADPH as an electron donor. The soluble diiron monooxygenases are usually composed of one or more components and are present in the cytoplasm of the cell. Soluble diiron monooxygenases include soluble methane monooxygenases, phenol hydroxylases, toluene monooxygenases, and alkene monooxygenases (See, Leahy et al., Evolution of the Soluble Diiron Monoxygenases, FEMS Microbiology Reviews, Vol. 27., p.449-479, 2003, which is incorporated by reference herein in its entirety, including any drawings).

As used herein, “soluble diiron monooxygenase system” refers to a system of proteins that comprises or consists of one or more subunits of a soluble diiron monooxygenase. As used herein, soluble diiron monooxygenase may also refer to, but need not, all components that increase the activity of the one or more subunits of the soluble diiron monooxygenase such as, for example, without limitation, one or more chaperone proteins such as one or more groEL2 proteins.

As used herein, “subunit” refers to protein molecule that assembles or coassembles with other protein molecules to form a protein complex or enzyme. The disclosure is intended to include some or all of the subunits from any microorganism or combination of microorganisms, as determined by one skilled in the art.

As used herein, “vector” refers a nucleic acid molecule used as a vehicle to artificially carry foreign genetic material into another cell, such as a host cell, where the foreign genetic material can be replicated and/or expressed in the host cell.

As used herein, “wild-type” or “WT” refers to a strain, gene, or characteristic that prevails among individuals in natural conditions. A wild-type may be contrasted against strains, genes, or characteristics that do not prevail among organisms in natural conditions, such as those from a mutant type.

### II. Soluble Diiron Monooxygenase

Some embodiments provide at least one mutant soluble diiron monooxygenase system comprising at least one mutant subunit, wherein the at least one mutant subunit comprises one or more mutations such that the activity of the at least one mutant soluble diiron monooxygenase system is greater than the activity of the wild-type soluble diiron monooxygenase system.

Methanotrophs consume methane as their major carbon source and have an essential role in the global carbon cycle by limiting escape of the greenhouse gas methane to the atmosphere. To oxidize methane to methanol, bacteria use soluble methane monooxygenases and particulate methane monooxygenases.

Bacterial multicomponent monooxygenases are exceptional in their ability to hydroxylate a broad spectrum of hydrocarbon substrates. Soluble monooxygenases, alkene mono-oxygenases, phenol hydroxylases, and alkene/aromatic monooxygenases belong to the bacterial multicomponent monooxygenase superfamily. Soluble diiron monooxygenases may also accept substrates other than alkanes, and in some cases, the enzymes might hydroxylate a wide range of molecules. One such substrate is the simplest alkene, ethylene (also known as ethene, C₂H₄), which can be converted into ethylene oxide. Similarly, propylene is converted into propylene oxide by soluble diiron monooxygenases. Soluble monooxygenases contain three protein components, a hydroxylase (MMOH, which is composed of three subunits, the alpha, beta, and gamma, as provided herein), a reductase (MMOR), and a regulatory protein (MMOB). MMOD and groEL2 are also required for optimal catalytic function.

Any mutant subunits disclosed herein may be used and may be mixed and matched with one another provided only that the at least one mutant soluble diiron monooxygenase system is improved. In some embodiments, the at least one mutant soluble diiron monooxygenase system comprises or consists one or more of mutant subunits disclosed herein. In some embodiments, the at least one mutant soluble diiron monooxygenase system comprises or consists of any one or more of a mutant hydroxylase alpha subunit, a mutant hydroxylase beta subunit, a mutant hydroxylase gamma subunit, a mutant accessory protein B, a mutant reductase subunit C, a mutant subunit D, and/or a mutant protein folding chaperone groEL-2.

In some embodiments, the at least one mutant soluble diiron monooxygenase system comprises or consists of at least one mutant subunit from one or more organisms. In some embodiments, the at least one mutant soluble diiron monooxygenase system comprises or consists of more than one subunits coming from one or more than one organism. In other words, any combination of mutant subunits from any organism may be used and the one or more organisms may be mixed and matched with one another provided only that the at least one mutant soluble diiron monooxygenase system is improved.

In some embodiments, the one or more organisms comprises or consists of one or more of Methylococcus capsulatus (Bath), Methylocystis sp. strain M, Methylosinus trichosporium OB3b, Rhodococcus rhodochrous B-276, Ralstonia pickettii PKO1, Pseudomonas mendocina KR1, Xanthobacter sp. Py2, Pseudomonas sp. strain JS150, and/or Pseudomonas sp. strain CF600. In some embodiments, the one or more organisms comprises or consists of one or more organisms from Table O.

TABLE O

| | Size | | % | % | % | | Accession |
|---|---|---|---|---|---|---|---|
| Gene | (aa) | Enzyme | Identity | Dissimilarity | Gaps | Organism | no. |
| mmoX | 527 | sMMO | — | — | — | Methylococcus | M90050 |
| | | | | | | capsulatus (Bath) | |
| mmoX | 526 | sMMO | 82.4 | 17.5 | 0.2 | Methylocystis sp. strain | U81594 |
| | | | | | | M | |
| mmoX | 525 | sMMO | 81.2 | 18.6 | 0.2 | Methylosinus | X55394 |
| | | | | | | trichosporium OB3b | |
| amoC | 501 | AMO | 34.2 | 61.6 | 4.3 | Rhodococcus | D37875 |
| | | | | | | rhodochrous B-276 | |
| tbuAl | 501 | T3MO | 17.9 | 74.2 | 7.9 | Ralstonia pickettii PKO1 | U04052 |
| tmoA | 500 | T4MO | 17.9 | 74.0 | 8.0 | Pseudomonas mendocina | M65106 |
| | | | | | | KR1 | |
| aamA | 497 | AMO | 17.9 | 73.8 | 8.2 | Xanthobacter sp. Py2 | AJ006979 |
| tbmD | 513 | T2MO | 17.4 | 74.8 | 7.8 | Pseudomonas sp. strain | L40033 |
| | | | | | | JS150 | |
| dmpN | 517 | PH | 17.0 | 73.7 | 9.3 | Pseudomonas sp. strain | M60276 |
| | | | | | | CF600 | |
| mmoY | 389 | sMMO | — | — | — | Methylococcus | M90050 |
| | | | | | | capsulatus (Bath) | |
| mmoY | 395 | sMMO | 60.0 | 38.5 | 1.5 | Methylocystis sp. strain | U81594 |
| | | | | | | M | |
| mmoY | 395 | sMMO | 58.7 | 39.7 | 1.5 | Methylosinus | X55394 |
| | | | | | | trichosporium OB3b | |
| amoA | 343 | AMO | 25.1 | 66.4 | 8.5 | Rhodococcus | D37875 |
| | | | | | | rhodochrous B-276 | |
| tbuA2 | 329 | T3MO | 15.4 | 78.5 | 6.1 | Ralstonia pickettii PKO1 | U04052 |
| dmpL | 331 | PH | 13.3 | 79.0 | 7.7 | Pseudomonas sp. strain | M60276 |
| | | | | | | CF600 | |
| tbmB | 336 | T2MO | 12.9 | 81.7 | 5.4 | Pseudomonas sp. strain | L40033 |
| | | | | | | JS150 | |
| tmoE | 327 | T4MO | 12.0 | 81.6 | 6.4 | Pseudomonas mendocina | M65106 |
| | | | | | | KR1 | |
| mmoB | 141 | sMMO | — | — | — | Methylococcus | M90050 |
| | | | | | | capsulatus (Bath) | |
| mmoB | 138 | sMMO | 65.7 | 32.9 | 1.4 | Methylocystis sp. strain | U81594 |
| | | | | | | M | |
| mmoB | 138 | sMMO | 65.7 | 32.9 | 1.4 | Methylosinus | X55394 |
| | | | | | | trichosporium OB3b | |
| tbuV | 104 | T3MO | 22.0 | 73.4 | 4.6 | Ralstonia pickettii PKO1 | U04052 |
| dmpM | 90 | PH | 20.6 | 72.2 | 7.2 | Pseudomonas sp. strain | M60276 |
| | | | | | | CF600 | |
| amoB | 117 | AMO | 20.2 | 74.2 | 5.6 | Rhodococcus | D37875 |
| | | | | | | rhodochrous B-276 | |
| tmoD | 103 | T4MO | 19.3 | 75.2 | 5.5 | Pseudomonas mendocina | M65106 |
| | | | | | | KR1 | |
| tbmC | 89 | T2MO | 18.8 | 74.0 | 7.3 | Pseudomonas sp. strain | L40033 |
| | | | | | | JS150 | |
| mmoC | 348 | sMMO | — | — | — | Methylococcus | M90050 |
| | | | | | | capsulatus (Bath) | |
| mmoC | 343 | sMMO | 50.1 | 47.8 | 2.0 | Methylocystis sp. strain | U81594 |
| | | | | | | M | |
| mmoC | 340 | sMMO | 41.8 | 51.4 | 6.8 | Methylosinus | S81887 |
| | | | | | | trichosporium OB3b | |
| amoD | 342 | AMO | 28.5 | 63.7 | 7.9 | Rhodococcus | D37875 |
| | | | | | | rhodochrous B-276 | |
| dmpP | 353 | PH | 25.8 | 65.8 | 8.4 | Pseudomonas sp. strain | M60276 |
| | | | | | | CF600 | |
| tmoF | 326 | T4MO | 25.5 | 64.8 | 9.7 | Pseudomonas mendocina | M95045 |
| | | | | | | KR1 | |
| tbmF | 355 | T2MO | 23.2 | 69.5 | 7.3 | Pseudomonas sp. strain | L40033 |
| | | | | | | JS150 | |
| tbuC | 334 | T3MO | 22.7 | 69.0 | 8.2 | Ralstonia pickettii PKO1 | U04052 |

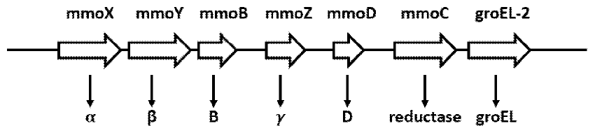

In some embodiments, the one or more organisms comprises or consists of Methylococcus capsulatus (Bath) (See, Coufal, D.; Blazyk, J; Whittington, D; Wu, W.; Rosensweig, A.; and Lippard, S., Sequencing and Analysis of the Methylococcus capsulatus (Bath) solumble methane monooxygenase genes (2000) Eur. J. Biochem. 267, 2174-2185, which is incorporated by reference in its entirety herein, including any drawings). One of the most well-studied methane monooxygenases is from Methylococcus capsulatus (Bath). (See, for example, Petroleum Biotechnology by Vazquez-Duhalt and Quintero-Romero in 2004, which is incorporated by reference herein in its entirety, including any drawings). FIG. 1 sets forth a schematic overview of the soluble methane monooxygenase operon of M. capsulatus.

### III. Hydroxylase

MMOH from M. capsulatus is approximately 245 kD and is a non-heme, oxo-bridged diiron catalytic cluster which catalyzes dioxygen-dependent oxidation-hydroxylation reactions within diiron centers. MMOH is a multi-subunit dimeric (α₂ß₂δ₂ or α₂ß₂) protein, where each α-subunit hosts the diiron center. The carboxylate-bridged diiron center is the locus for O₂ activation and subsequent substrate hydroxylation/epoxidation. The diiron center is similar to those in the R2 subunit of ribonucleotide reductase, ferritin, stearoyl acyl carrier protein, desaturase, and the aging-related protein Clk1.

In some embodiments, the at least one mutant soluble diiron monooxygenase system comprises a mutant hydroxylase alpha subunit. In some embodiments, the at least one mutant soluble diiron monooxygenase system comprises a mutant hydroxylase beta subunit. In some embodiments, the at least one mutant soluble diiron monooxygenase system comprises a mutant hydroxylase gamma subunit.

The hydroxylase alpha, hydroxylase beta, and hydroxylase gamma proteins are also referred to as MMOX, MMOY, MMOZ, respectively. The hydroxylase alpha protein from M. capsulatus is approximately 60.6 kD. The hydroxylase beta protein from M. capsulatus is approximately 45.1 kD. The hydroxylase gamma protein from M. capsulatus is approximately 19.8 kD. The proteins are respectively encoded by the mmoX, mmoY, and mmoZ genes.

### IV. The Regulatory Protein (Accessory Protein B)

In some embodiments, the at least one mutant soluble diiron monooxygenase system comprises a mutant accessory protein B. Accessory protein B from M. capsulatus is a 15.8 kD protein that acts without co-factors. The MMOB/DmpM family comprises monooxygenase components such as the methane monooxygenase (EC:1.14.13.25) regulatory protein B. The family also includes DmpM or Phenol hydroxylase (EC:1.14.13.7) protein component P2, a protein that lacks redox co-factors and is required for optimal turnover of phenol hydroxylase. When the accessory protein B is present at a low concentration, it converts methane monooxygenase from an oxidase to a hydroxylase and stabilizes intermediates required for the activation of dioxygen.

Accessory protein B is required to couple electron consumption with substrate hydroxylation at the catalytic diiron center of the hydroxylase. When accessory protein B binds the hydroxylase, it triggers simultaneous conformational changes that modulate oxygen and methane access as well as proton delivery to the diiron center. Accessory protein B binds to the “canyon” of the hydroxylase (Asp 36-Leu 129) and competitively inhibits binding of reductase subunit C.

### V. The Reductase

In some embodiments, the at least one mutant soluble diiron monooxygenase system comprises a mutant reductase subunit C.

Reductase subunit C from M. capsulatus is a 38.4 kD protein. Electron transfer to the diiron(III) centers in the hydroxylase involves NADH reductions of an oxidized FAD cofactor to a hydroquinone form, followed by the ferredoxin domain sequentially shuttling two electrons from the reduced FAD cofactor to the diiron center in the hydroxylase (See, Electron Transfer Control in Soluble Methane Monooxygenase; Weixue Wang, Roxana E. Iacob, Rebecca P. Luoh, John R. Engen, and Stephen J. Lippard; J. Am. Chem. Soc., 2014, 136 (27), pp 9754-9762, which is incorporated in its entirety herein, including any drawings). The isolated domains have stable structures and retain biochemical properties of the two domains in full-length reductase subunit C.

### VI. Subunit D, MMOD

In some embodiments, the at least one mutant soluble diiron monooxygenase system comprises a mutant MMOD or mutant subunit D. The cluster of genes that encode for the three characterized protein components of the hydroxylase, accessory protein subunit B, and the reductase subunit C also contains an additional open reading frame, orfY, which codes for a protein, MMOD, of unknown function. Despite the fact that there is no known function of MMOD, mutations in MMOD can improve the activity of a monooxygenase. MMOD is expressed in a native organism although at significantly lower levels that other monooxygenase proteins. Cofactorless MMOD is a potent inhibitor of soluble monooxygenase activity and binds to hydroxylase with a similar affinity to that of accessory protein subunit B and the reductase subunit C.

### VII. GroEL-2

In some embodiments, the at least mutant one soluble diiron monooxygenase system comprises a mutant protein folding chaperone groEL-2. GroEL-2 belongs to the chaperonin family of molecular chaperones and is found in a large number of bacteria. The groEL-2 gene is often located in close proximity to the monooxygenase operon and groEL-2 probably has a special relationship to the methane monooxygenase. GroEL-2 is thought to be required for the proper folding of many proteins and some bacteria, including M. capsulatus, have more than one gene homologous to groEL-2. Within the cell, the process of groEL-2-mediated protein folding involves multiple rounds of binding, encapsulation, and release of substrate protein.

### VIII. Directed Evolution

In some embodiments, the activity of the at least one mutant soluble diiron monooxygenase system is improved through directed evolution. Any method of directed evolution well-known to one skilled in the art is within the scope of the invention.

Directed evolution consists of iterations of three steps; generating genetic diversity, assaying (screening or selecting) the diversity for a property of interest to identify beneficial, neutral, and/or deleterious mutations, and recombining a subset of mutations which can then be screened for improved mutants. Genetic variants may be used as templates either for additional rounds of recombination of the subset of mutations or for the discovery of additional genetic diversity of the at least one mutant soluble diiron monooxygenase system. The methods used to generate genetic diversity, to assay the at least one mutant soluble diiron monooxygenase system, and to recombine the mutations may vary.

Many methods are available for the generation of genetic diversity in a DNA sequence; chemical mutagenesis, ultraviolet-light-induced mutagenesis, error-prone PCR, directed saturation mutagenesis, and others. Any combination of these methods may also be used according to the invention. In some embodiments, each mutation is measured for its effects on the activity of at least one mutant soluble diiron monooxygenase system, which may be measured along one or more features or dimensions. For example, amino acid sequence variants of the protein(s) can be prepared by mutations in the DNA. Methods for mutagenesis and nucleotide sequence alterations include, for example, Kunkel, (1985) Proc Natl Acad Sci USA 82;488-92; Kunkel, et al., (1987) Meth Enzymol 154;367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff, et al., (1978) Atlas of Protein Sequence and Structure (Natl Biomed Res Found, Washington, D.C.). Each of the above-cited references is incorporated by reference in its entirety herein, including any drawings.

Furthermore, any of the one or more nucleic acids or one or more vectors (or any others mentioned herein or any of the regulatory elements that control or modulate expression thereof) disclosed herein may be optimized by genetic/protein engineering techniques, such as directed evolution or rational mutagenesis. Directed evolution allows one of ordinary skill in the art to optimize the enzymes for expression and activity in yeast, bacteria, or any other suitable cell or organism.

Directed evolution is exemplified herein to discover mutations for soluble methane monooxygenase from Methylococcus capsulatus (Bath) when functionally expressed inside E. coli cells. As set forth in the examples, a large mutant library was cloned and assayed using a non-fluorescent substrate that became fluorescent when hydroxylated by the activity of the enzyme. Any mutant that displayed improved activity was reassayed using the same and/or additional methods or conditions. Improved variants were then subjected to DNA sequencing to identify the mutations that caused increased activity.

Once a group of clones was identified as having improved activity, clones were sequenced to identify mutation(s) that were generated. DNA can be sequenced using any technique known to one skilled in the art, such as, for example, without limitation, Sanger sequencing. Higher-throughput techniques, such as those offered by Illumina or Pacific Biosciences, may also be utilized.

In some embodiments, combinations of mutations that display desirable activity are combined to form at least one mutant soluble diiron monooxygenase system that has particularly favorable activity. Once the recombined mutants have been constructed, one can assay these variants using the same techniques that were used previously to assay the original mutant libraries or using other assays that measure the at least one mutant soluble diiron monooxygenase system's properties. The combinations can be tested either by deliberately constructing specifically desired clones or by recombining mutations randomly in a one pot reaction. Methods of DNA construction for recombination libraries are well-known to those skilled in the art and include a variety of techniques, including SOE PCR, transfer PCR, and Quikchange mutagenesis (Agilent Technologies).

### IX. Nucleic Acids and Vectors and Integration

Some aspects provide one or more isolated nucleic acids encoding any of the soluble diiron monooxygenase systems provided herein. In some embodiments, the one or more nucleic acids comprise one or more vectors. In some embodiments, the one or more nucleic acids are integrated into host cells.

The one or more nucleic acids can be introduced into the one or more host cells by any method known to one of skill in the art without limitation (See, for example, Hinnen et al. (1978) Proc. Natl. Acad. Sci. USA 75;1292-3; Cregg et al. (1985) Mol. Cell. Biol. 5;3376-3385; Goeddel et al. eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY, each of which is incorporated by reference in its entirety herein, including any drawings). Exemplary techniques include, but are not limited to, spheroplasting, electroporation, PEG 1000 mediated transformation, and lithium acetate or lithium chloride mediated transformation. In some embodiments, the one or more vectors comprises or consists of an extrachromosomal plasmid. In some embodiments, the one or more vectors comprises or consists of a chromosomal integration vector that can integrate the nucleotide sequence into the chromosome of the one or more host cells.

Expression of the one or more nucleic acids or one or more vectors may be modified. For example, the copy number of the one or more nucleic acids or one or more vectors may be altered by modifying transcription. This can be achieved, for example, by modifying the copy number of the nucleotide sequence of the one or more nucleic acids or one or more vectors (e.g., by using or generating a higher or lower copy number expression vector comprising the nucleotide sequence, by introducing additional copies of the nucleotide sequence into the genome of the one or more host cells cell, or by genetically modifying, deleting, or disrupting the nucleotide sequence in the genome of the one or more host cells) by changing the order of coding sequences on a polycistronic mRNA of an operon, or by breaking up an operon into individual genes, each with its own control elements. The strength of a promoter, enhancer, or operator to which a nucleotide sequence is operably linked may also be manipulated, increased, decreased, or different promoters, enhancers, or operators may be introduced.

Alternatively, or in addition, the copy number of the one or more nucleic acids or one or more vectors may be altered by modifying the level of translation of an mRNA. This can be achieved, for example, by modifying the stability of the mRNA, modifying the sequence of the ribosome binding site, modifying the distance or sequence between the ribosome binding site and the start codon of the enzyme coding sequence, modifying the entire intercistronic region located “upstream of” or adjacent to the 5′ side of the start codon of the enzyme coding region, stabilizing the 3′-end of the mRNA transcript using hairpins and specialized sequences, modifying the codon usage of an enzyme, altering expression of rare codon tRNAs used in the biosynthesis of the enzyme, and/or increasing the stability of an enzyme, as, for example, via mutation of its coding sequence.

Expression of the one or more nucleic acids or one or more vectors may be modified or regulated by targeting particular genes. For example, without limitation, in some embodiments of the methods described herein, the one or more host cells is contacted with one or more nucleases capable of cleaving, i.e., causing a break at a designated region within a selected site. In some embodiments, the break is a single-stranded break, that is, one but not both strands of the target site is cleaved. In some embodiments, the break is a double-stranded break. In some embodiments, a break inducing agent is any agent that recognizes and/or binds to a specific polynucleotide recognition sequence to produce a break at or near a recognition sequence. Examples of break inducing agents include, but are not limited to, endonucleases, site-specific recombinases, transposases, topoisomerases, and zinc finger nucleases, and include modified derivatives, variants, and fragments thereof.

In some embodiments, the recognition sequence within a selected target site can be endogenous or exogenous to a cell's genome. When the recognition site is an endogenous or exogenous sequence, it may be a recognition sequence recognized by a naturally occurring or native break inducing agent. Alternatively, an endogenous or exogenous recognition site could be recognized and/or bound by a modified or engineered break inducing agent designed or selected to specifically recognize the endogenous or exogenous recognition sequence to produce a break. In some embodiments, the modified break inducing agent is derived from a native, naturally occurring break inducing agent. In other embodiments, the modified break inducing agent is artificially created or synthesized. Methods for selecting such modified or engineered break inducing agents are known in the art.

In some embodiments, the one or more nucleases is a CRISPR/Cas-derived RNA-guided endonuclease. CRISPR may be used to recognize, genetically modify, and/or silence genetic elements at the RNA or DNA level or to express heterologous or homologous genes. CRISPR may also be used to regulate endogenous or exogenous nucleic acids. Any CRISPR/Cas system known in the art finds use as a nuclease in the methods and compositions provided herein. CRISPR systems that find use in the methods and compositions provided herein also include those described in International Publication Numbers WO 2013/142578 A1, WO 2013/098244 A1 and Nucleic Acids Res (2017) 45 (1); 496-508, the contents of which are hereby incorporated in their entireties).

In some embodiments, the one or more nucleases is a TAL-effector DNA binding domain-nuclease fusion protein (TALEN). TAL effectors of plant pathogenic bacteria in the genus Xanthomonas play important roles in disease, or trigger defence, by binding host DNA and activating effector-specific host genes. (See, e.g., Gu et al. (2005) Nature 435:1122-5; Yang et al., (2006) Proc. Natl. Acad. Sci. USA 103:10503-8; Kay et al., (2007) Science 318:648-51; Sugio et al., (2007) Proc. Natl. Acad. Sci. USA 104:10720-5; Romer et al., (2007) Science 318:645-8; Boch et al., (2009) Science 326(5959):1509-12; and Moscou and Bogdanove, (2009) 326(5959):1501, each of which is incorporated by reference in their entirety). A TAL effector comprises a DNA binding domain that interacts with DNA in a sequence-specific manner through one or more tandem repeat domains. The repeated sequence typically comprises 34 amino acids, and the repeats are typically 91-100% homologous with each other. Polymorphism of the repeats is usually located at positions 12 and 13, and there appears to be a one-to-one correspondence between the identity of repeat variable-diresidues at positions 12 and 13 with the identity of the contiguous nucleotides in the TAL-effector's target sequence.

The TAL-effector DNA binding domain may be engineered to bind to a desired sequence and fused to a nuclease domain, e.g., from a type II restriction endonuclease, typically a nonspecific cleavage domain from a type II restriction endonuclease such as FokI (See, e.g., Kim et al. (1996) Proc. Natl. Acad. Sci. USA 93:1156-1160, which is incorporated by reference in its entirety herein, including any drawings). Other useful endonucleases may include, for example, HhaI, HindIII, Nod, BbvCI, EcoRI, BglI, and AlwI. Thus, in preferred embodiments, the TALEN comprises a TAL effector domain comprising a plurality of TAL effector repeat sequences that, in combination, bind to a specific nucleotide sequence in a target DNA sequence, such that the TALEN cleaves the target DNA within or adjacent to the specific nucleotide sequence. TALENS useful for the methods provided herein include those described in WO10/079430 and U.S. Patent Application Publication No. 2011/0145940, which is incorporated by reference herein, including any drawings.

In some embodiments, the one or more of the nucleases is a zinc-finger nuclease (ZFN). ZFNs are engineered break inducing agents comprised of a zinc finger DNA binding domain and a break inducing agent domain. Engineered ZFNs consist of two zinc finger arrays each of which is fused to a single subunit of a non-specific endonuclease, such as the nuclease domain from the FokI enzyme, which becomes active upon dimerization.

Useful zinc-finger nucleases include those that are known and those that are engineered to have specificity for one or more sites. Zinc finger domains are amenable for designing polypeptides that specifically bind a selected polynucleotide recognition sequence. Thus, they are amenable to modifying or regulating expression by targeting particular genes.

The one or more nucleic acids or one or more vectors can be modified in a number of other ways, including, but not limited to, gene silencing or any other form of genetic modification, expressing a modified form of the one or more nucleic acids or one or more vectors that exhibits increased or decreased solubility in the one or more host cells, expressing an altered form of the expressed one or more nucleic acids or one or more vectors that lacks a domain through which the activity of the enzyme is inhibited, expressing a modified form of the expressed one or more nucleic acids or one or more vectors that has a higher or lower Kcat or a lower or higher Km for a substrate, or expressing one or more polypeptides from the one or more nucleic acids or one or more vectors that is more or less affected by feed-back or feed-forward regulation by another molecule in the pathway.

In some embodiments, the at least one mutant soluble diiron monooxygenase system is modified. It will be recognized by one skilled in the art that absolute identity to the at least one mutant soluble diiron monooxygenase system is not strictly necessary. For example, changes in a particular gene or polynucleotide comprising a sequence encoding a polypeptide or an enzyme can be performed and screened for activity. Such modified or mutated polynucleotides and polypeptides can be screened for expression or function using methods known in the art.

Those of skill in the art will recognize that, due to the degenerate nature of the genetic code, a variety of polynucleotides differing in their nucleotide sequences can be used for the at least one mutant soluble diiron monooxygenase system of the disclosure. Due to the inherent degeneracy of the genetic code, other polynucleotides that encode substantially the same or functionally equivalent polypeptides or subunits can also be used. The disclosure includes polynucleotides of any sequence that encode the amino acid sequences of the polypeptides and proteins of the enzymes utilized in the methods of the disclosure.

In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have an activity that is identical or similar to the referenced polypeptide. Accordingly, the at least one mutant soluble diiron monooxygenase system encoded by the one or more nucleic acids or one or more vectors shown herein merely illustrate embodiments of the disclosure.

The disclosure also includes at least one mutant soluble diiron monooxygenase system with different amino acid sequences than the specific monooxygenase systems described herein if the modified or variant polypeptides have an activity that is desirable yet different from referenced polypeptide. In some embodiments, the at least one mutant soluble diiron monooxygenase system may be altered by modifying the gene that encodes the at least one mutant soluble diiron monooxygenase system so that the expressed systems proteins are more or less active than the wild-type version.

As will be understood by those of skill in the art, it can be advantageous to modify a coding sequence to enhance expression in a particular host or one or more host cells. The genetic code is redundant with 64 possible codons, but most organisms typically use a subset of these codons. Codons can be substituted to reflect the preferred codon usage of the host, in a process sometimes called “codon optimization” or “controlling for species codon bias.”

Optimized coding sequences containing codons preferred by a particular prokaryotic or eukaryotic hosts (See, for example, Murray et al., 1989, Nucl Acids Res. 17: 477-508, which is incorporated by reference in its entirety herein, including any drawings) can be prepared, for example, to increase the rate of translation or to produce recombinant RNA transcripts having desirable properties, such as a longer half-life, as compared with transcripts produced from a non-optimized sequence. Translation stop codons can also be modified to reflect preference in the one or more host cells. For example, typical stop codons for S. cerevisiae and mammals are UAA and UGA, respectively.

In addition, homologs of the at least one mutant soluble diiron monooxygenase system are encompassed by the disclosure. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

It is recognized that residue positions that are not identical often differ by conservative amino acid substitutions. In cases where two or more amino acid sequences differ from each other by conservative substitutions, the percent sequence identity or degree of homology may practically be adjusted upwards to correct for the conservative nature of the substitution. Means for making this adjustment are well known to those of skill in the art (See, e.g., Pearson W. R., 1994, Methods in Mol Biol 25: 365-89, which is incorporated by reference in its entirety herein, including any drawings).

Sequence homology and sequence identity for polypeptides is typically measured using sequence analysis software. A typical algorithm used comparing a molecule sequence to a database containing a large number of sequences from different organisms is the computer program BLAST. When searching a database containing sequences from a large number of different organisms, it is typical to compare amino acid sequences.

In addition, genes encoding enzymes homologous to the polypeptides encoded by the one or more nucleic acids or one or more vectors can be identified from other fungal and bacterial species or other species if they are orthologous or if there is homology between the two chosen species. Sources of prokaryotic enzymes that are useful include, but are not limited to, Methylococcus capsulatus (Bath), Methylocystis sp. strain M, Methylosinus trichosporium OB3b, Rhodococcus rhodochrous B-276, Ralstonia pickettii PKO1, Pseudomonas mendocina KR1, Xanthobacter sp. Py2, Pseudomonas sp. strain JS150, and/or Pseudomonas sp. strain CF600 or any of the one or more organisms from Table O.

Techniques known to those skilled in the art may be suitable to identify additional homologous genes and homologous enzymes. Generally, analogous genes and/or analogous enzymes can be identified by functional analysis and will have functional similarities. As an example, to identify homologous or analogous biosynthetic pathway genes, proteins, or enzymes, techniques may include, but are not limited to, cloning a gene by PCR using primers based on a published sequence of a gene/enzyme of interest or by degenerate PCR using degenerate primers designed to amplify a conserved region among a gene of interest.

Further, one skilled in the art can use other techniques to identify homologous or analogous genes, proteins, or enzymes with functional homology or similarity to any of mutant soluble diiron monooxygenase systems disclosed herein. Techniques include examining a cell or cell culture for catalytic activity (e.g. as described herein or in Kiritani, K., Branched-Chain Amino Acids Methods Enzymology, 1970, which is incorporated by reference in its entirety herein, including any drawings), then isolating the enzyme with the activity through purification, determining the protein sequence of the enzyme through techniques such as Edman degradation, design of PCR primers to the likely nucleic acid sequence, amplification of the DNA sequence through PCR, and cloning of the nucleic acid sequence. To identify homologous or similar genes and/or homologous or similar proteins, analogous genes and/or analogous proteins, techniques also include comparison of data concerning a candidate gene or enzyme with databases such as BRENDA, KEGG, or MetaCYC.

In some embodiments, the at least one mutant soluble diiron monooxygenase system comprises or consists of a mutant subunit having homology to any of the subunits disclosed herein. In some embodiments, the at least one mutant soluble diiron monooxygenase system comprises or consists of a mutant subunit having homology of at least about 80%, at least about 85%, at least about 90%, at least about 91%, at least about 92%, at least about 93%, at least about 94%, at least about 95%, at least about 96%, at least about 97%, at least about 98%, or at least about 99% homology to any of the mutant subunits disclosed herein. In some embodiments, the at least one mutant soluble diiron monooxygenase system comprises or consists of a system having homology of at least about 80%, at least about 85%, at least about 90%, at least about 91%, at least about 92%, at least about 93%, at least about 94%, at least about 95%, at least about 96%, at least about 97%, at least about 98%, or at least about 99% homology to any one or more of the subunits or groups of proteins disclosed herein.

The invention is not just limited to subunits and systems of M. capsulatus and subunits and systems other than those from M. capsulatus are intended to be within the scope of the invention. In some embodiments, the at least one mutant soluble diiron monooxygenase system comprises or consists of a mutant subunit from a species other than M. capsulatus having homology to any of the subunits disclosed herein. In some embodiments, the at least one mutant soluble diiron monooxygenase system comprises or consists of a mutant subunit from a species other than M. capsulatus having homology of at least about 80%, at least about 85%, at least about 90%, at least about 91%, at least about 92%, at least about 93%, at least about 94%, at least about 95%, at least about 96%, at least about 97%, at least about 98%, or at least about 99% homology to any of the mutant subunits disclosed herein. In some embodiments, the at least one mutant soluble diiron monooxygenase system comprises or consists of a system from a species other than M. capsulatus having homology of at least about 80%, at least about 85%, at least about 90%, at least about 91%, at least about 92%, at least about 93%, at least about 94%, at least about 95%, at least about 96%, at least about 97%, at least about 98%, or at least about 99% homology to any one or more of the subunits or groups of proteins disclosed herein.

### X. Cells or Host Cells

Cells that can be used may be any cells deemed useful by those of skill in the art. Cells useful in the compositions and methods provided herein include archaeal, prokaryotic, or eukaryotic cells. The term cell and/or host cells may be interchangeable as used throughout this specification. Either a cell or host cell may be modified or unmodified and may comprise a host that is unmodified and has a native pathway.

In some embodiments, the one or more host cells comprises or consists of one or more prokaryotic cells. In some embodiments, the one or more host cells comprises or consists of any one of gram-positive, gram-negative, and/or gram-variable bacteria. Examples include, but are not limited to, cells belonging to the genera: Actinobacillus, Agrobacterium, Alicyclobacillus, Anabaena, Anacystis, Anaerobiospirillum, Arthrobacter, Azobacter, Bacillus, Brevibacterium, Chromatium, Clostridium, Corynebacterium, Enterobacter, Erwinia, Escherichia, Gluconohacter, Klebsiella, Lactobacillus, Lactococcus, Mannheimia, Mesorhizobium, Methylobacterium, Microbacterium, Phormidium, Pseudomonas, Rhizobium, Rhodobacter, Rhodopseudomonas, Rhodospirillum, Rhodococcus, Salmonella, Scenedesmun, Serratia, Shigella, Staphlococcus, Streptomyces, Synnecoccus, and Zymomonas. Examples of strains include, but are not limited to: Actinobacillus succinogenes, Anaerobiospirillum succiniciproducens, Bacillus subtilis, Bacillus amyloliquefacines, Brevibacterium ammoniagenes, Brevibacterium immariophilum, Clostridium acetobutylicum, Clostridium beigerinckii, Enterobacter sakazakii, Escherichia coli, Gluconobacter oxydans, Klebsiella oxytoca, Lactobacillus plantarum, Lactococcus lactis, Mannheimia succiniciproducens, Mesorhizobium loti, Pseudomonas aeruginosa, Pseudomonas mevalonii, Pseudomonas fluorescens, Pseudomonas pudica, Rhizobium etli, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, Staphylococcus aureus, Streptomyces coelicolor, and Zymomonas mobilis.

In some embodiments, the one or more host cells comprises or consists of archaeal cells. In some embodiments, archaeal cells include, but are not limited to: Aeropyrum, Archaeglobus, Halobacterium, Methanococcus, Methanobacterium, Pyrococcus, Sulfolobus, and Thermoplasma. Examples of archaea strains include, but are not limited to: Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

In some embodiments, the one or more host cells comprises or consists of eukaryotic cells. In some embodiments, the eukaryotic cells include, but are not limited to, fungal cells, algal cells, insect cells, and plant cells. In some embodiments, yeasts useful in the present methods including yeasts that belong to the genera Aciculoconidium, Ambrosiozyma, Arthroascus, Arxiozyma, Ashbya, Aspergillus, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the one or more host cells comprises or consist of at least one of Escherichia coli, Bacillus subtilis, Bacillus methanolicus, Pseudomonas putida, Saccharomyces cerevisiae, Pichia pastoris, Pichia methanolica, Salmonella enterica, Corynebacterium glutamicum, Klebsiellct oxytoca, Anaerobiospirillum succiniciproducens, Actinobacillus succinogenes, Mannheimia succiniciproducens, Rhizobium etli, Gluconobacter oxydans, Zymomonas mobilis, Lactococcus lactis, Lactobacillus plantarum, Streptomyces coelicolor, Clostridium acetobutylicum, Pseudomonas fluorescens, Schizosaccharomyces pombe, Kluyveromyces lactis, Kluyveromyces marxianus, Aspergillus terreus, Aspergillus niger, and Candida utilis. In some embodiments, the one or more host cells comprises or consist of Escherichia coli. In some embodiments, the one or more host cells comprises or consists of Pichia pastoris. In some embodiments, the one or more host cells comprises or consists of Saccharomyces cerevisiae. In some embodiments, the one or more host cells comprises or consist of Corynebacterium glutamicum. In some embodiments, the one or more host cells comprises or consist of Bacillus methanolicus.

### XI. Chemicals

In some embodiments, the at least one mutant soluble diiron monooxygenase system is expressed in a host cell for production or one or more chemicals. In some embodiments, the one or more chemicals comprises or consists of one or more of dicarboxylic acid, malic acid, fumaric acid, succinic acid, malic acid salt, fumaric acid salt, succinic acid salt, L-malic acid, D-malic acid, maleic acid, lactic acid, adipic acid, 1,3-propanediol, 2,3-butanediol, 1,4-butanediol, butadiene, fatty acid derivatives, fatty alcohols, fatty acids, fatty acid esters, fatty acid methyl esters, fatty acid ethyl esters, branched fatty acids, branched fatty acid derivatives, omega-3 fatty acids, isoprenoids, isoprene, farnesene, farnesane, squalene, squalane, carotenoids, any or all of the amino acids, alanine, arginine, asparagine, aspartic acid, cysteine, glutamic acid, monosodium glutamate, glutamine, glycine, histidine, isoleucine, leucine, lysine, methionine, phenylalanine, threonine, tryptophan, valine, ornithine, proline, selenocysteine, serine, tyrosine, epoxides such as ethylene oxide and propylene oxide, ethanol, propanol, 1-butanol, 2-butanol, isobutanol (2-methylpropan-1-ol), alcohols, alkanes, alkenes, olefins, animal feed additives, mixtures of amino acids, and proteins. Other examples of chemicals include, but are not limited to, ethanol, propanol, isopropanol, butanol, fatty alcohols, fatty acid esters, ethyl esters, wax esters; hydrocarbons and alkanes such as propane, octane, diesel, Jet Propellant 8 (JP8); terephthalate, 1,3-propanediol, 1,4-butanediol, acrylate, adipic acid, c-caprolactone, isoprene, caprolactam, and polymers of these, plus other polymers, such as polyols, polyhydroxyalkanoates (PHA), poly-beta-hydroxybutyrate (PHB), rubber; commodity chemicals such as lactate, docosahexaenoic acid (DHA), 3-hydroxypropionate, y-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, 3-hydroxypropionic acid (HPA), lactic acid, THF, gamma butyrolactone, pyrrolidones, hydroxybutyrate, glutamic acid, levulinic acid, acrylic acid, malonic acid, chemicals such as carotenoids, isoprenoids, itaconic acid; pharmaceuticals and pharmaceutical intermediates such as 7-aminodeacetoxycephalosporanic acid (7-ADCA)/cephalosporin, erythromycin, polyketides, statins, paclitaxel, docetaxel, terpenes, peptides, steroids, omega fatty acids and other such suitable products of interest.

In some embodiments, the one or more chemicals comprises or consists of biofuels, industrial and specialty chemicals, intermediates used to make additional products, nutritional supplements, nutraceuticals, polymers, paraffin replacements, personal care products, and pharmaceuticals. In some embodiments, the one or more chemicals comprises or consists of natural and non-natural compounds such as, for example, amino acids, nucleic acids, nucleotides, polynucleotides and all related biological molecules, amino acids and nucleotides that are modified in a way differently than they are normally modified in biological systems (such as, for example, without limitation, non-natural amino acids).

In some embodiments, the one or more chemicals comprises or consists of methanol, ethanol, propanol, butanol, or naphthol. In some embodiments, the one or more chemicals comprises or consists of succinate, malate, fatty acids, lysine, and/or glutamate. In an embodiment, the one or more chemicals comprises or consist of 3-hydroxypropionate or a polymer of 3-hydroxypropionate. In some embodiments, the one or more chemicals consists of methanol.

In some embodiments, the at least one mutant soluble diiron monooxygenase system comprises one or more mutant subunits as set forth herein, wherein the one or more mutant subunits is purified in vitro. In some embodiments, the at least one mutant soluble diiron monooxygenase system can be used in vitro to produce one or more chemicals.

### EXAMPLES

### Example 1: Mutagenesis of the Hydroxylase Alpha Subunit

A saturation library of mutations in the mmoX gene in pBZ34 (SEQ ID NO: 1) was constructed using degenerate primers. The library was transformed into a strain of E. coli named NH283 (derived from the commercially available strain NEB Express via a deletion of the araBAD operon using a chloramphenicol resistance gene and the method of Datsenko and Wanner (2000)). The library was recovered in SOC for 1 hour at 37° C., 280 rpm, and then spread on LB agar plates supplemented with 100 μg/mL spectinomycin.

Colonies were picked into 96-well deep-well plates (Costar), containing 400 μL LB supplemented with 1001.μg/mL spectinomycin. The plates were sealed with an air-permeable sealing tape and incubated shaking at 37° C., 270 rpm for about 16 hours. Each plate contained several wells with the strain BZ85 (NH283/pBZ34) as an on-plate control, against which all mutant colonies could be compared. After the 16-hour incubation, each well was subcultured into a fresh 96-well deep-well plate, using 25 μL inoculum into 250 μL of LB supplemented with 100 μg/mL spectinomycin, 1 mM L-arabinose, 50 μM ferric citrate, and 200 μM L-cysteine. These plates were sealed and incubated at 37° C., 270 rpm.

After 3-4 hours, 200 μL from each plate was sampled into a shallow-well plate and centrifuged at 4000 rpm for 6 min. The supernatant was removed and 225 μL of phosphate-buffered saline (PBS) was added to each well. Each plate was centrifuged again and the supernatant was removed. After a second PBS wash, 200 μL of PBS supplemented with 5 mM coumarin, 0.4% glycerol, and 50 μM ferric citrate was added to each well. The plates were centrifuged again, covered to prevent evaporation, and placed at 37° C. for 16 hours. Then, each plate was measured for fluorescence by excitation at 360 nm and emission at 460 nm.

Using the fluorescence readings, improved clones were identified by the following method. For each plate, the fluorescence in each well was normalized by the average value of the fluorescence in all the control wells (containing BZ85). Clones that demonstrated significant increases in fluorescence relative to the control were selected for a second fluorescence screen to confirm the improved enzyme activity. From this set, any clone that was above a desirable threshold was sequenced to identify the mutation that led to improved activity. Additional assays may also be performed to test other features of the enzyme.

A subset of the clones was selected for DNA sequencing to identify mutations that improve the activity of the enzyme. Table 1 shows the mutation(s) that was identified, along with the relative activity (activity normalized by controls) in the coumarin assay, the positions being numbered with respect to SEQ ID NO: 3.

TABLE 1
Mutations in the hydroxylase alpha subunit.

| Mutation(s) | Activity |
|---|---|
| L003P | 1.38 |
| L003P, L014P | 3.81 |
| L003P, L014P, L032P, F035L, | 2.78 |
| R330T | |
| L003P, L014P, S434N | 1.91 |
| A025S | 1.74 |
| Q026G | 1.20 |
| L032P, A262E | 1.24 |
| Q033K | 1.47 |
| Q033R | 1.17 |
| N042R, T044A | 1.25 |
| T044S | 1.21 |
| A053G | 1.28 |
| N054K | 1.49 |
| N054L | 1.86 |
| E055A | 1.30 |
| E055S, N375H | 1.11 |
| T056A | 1.37 |
| T056G | 1.72 |
| K057G | 1.15 |
| K057S | 1.33 |
| K057T | 1.11 |
| K061A | 2.25 |
| K061R | 1.98 |
| A130C | 2.49 |
| A130G, R419L | 2.01 |
| A130H | 1.78 |
| A130T | 1.66 |
| N135S | 1.24 |
| V154L | 1.80 |
| V154L, S263Q | 1.71 |
| G162C | 2.86 |
| Q163F | 1.26 |
| H168G | 1.43 |
| L216M | 1.12 |
| Y251M | 1.10 |
| Y251W | 1.24 |
| A264Q | 1.44 |
| T277G | 1.34 |
| I316S | 1.14 |
| G322N | 1.32 |
| Y347G | 1.32 |
| T356C | 1.91 |
| T356G | 2.27 |
| T356M | 1.60 |
| A374E | 1.41 |
| D381R | 1.56 |
| D381S | 2.38 |
| Y387I | 1.57 |
| Y387L | 2.05 |
| R393C | 1.33 |
| S399D | 1.11 |
| L405G | 1.63 |
| R419S | 1.53 |
| V420C | 1.43 |
| Q422E, K431E, E480G, D504E | 1.27 |
| K431E | 1.34 |
| G432A | 1.84 |
| A433G, A525Q | 1.32 |
| A433Q | 1.48 |
| A433T | 1.40 |
| S434G, K505E | 1.22 |
| S434N | 1.61 |
| Q444E | 1.31 |
| G453V | 2.07 |
| M456V | 1.44 |
| A459E, K513T | 1.27 |
| I469V | 1.50 |
| F470I, G475* | 1.43 |
| L485E | 1.43 |
| K493E | 1.41 |
| K493V | 1.30 |
| K505E | 1.28 |
| D510E | 1.23 |
| K513R | 1.25 |
| K513T | 1.16 |
| L515G | 1.89 |
| F526C | 1.33 |
| F526G | 1.27 |
| F526L | 1.33 |
| F526S | 1.46 |

The following describes the method for culturing the strains and measuring the bioconversion of methane to methanol. All strains were inoculated in 1 mL LB Miller supplemented with spectinomycin (100 μg/mL) and grown at 37° C. for 18 hours with shaking at 280 rpm. The cultures grew to stationary phase and 0.1 mL of these cultures was then used to inoculate two flasks containing sterile 10 mL LB supplemented with spectinomycin (100 μg/mL), arabinose (1 mM), L-cysteine (200 μM), and ferric citrate (50 M). The cultures were grown with shaking at 37° C. until OD600˜1.2 (approximately 4.0-4.5 hours). The cells were spun for 5 minutes at 4000 rpm, and re-suspended in 10 mL phosphate buffer solution (PBS). This 10 mL was split equally into two glass serum bottles, 5 mL in each. The bottles were then sealed with butyl rubber stoppers. A volume of 60 mL of either methane or air was measured into syringes and injected through the stopper and into each of the two bottles. The bottles were shaken at 37° C. for 16-25 hours, at which point the supernatant was sampled in order to measure methanol concentration.

Methanol was measured using a colorimetric assay (Cell Biolabs catalog number STA-620). Briefly, it measures methanol using an enzymatic reaction that produces hydrogen peroxide, which reacts with a colorimetric probe. 90 μL of a reaction mixture was combined with 10 μL of sample and incubated at 37° C. for 30 minutes. The composition of the assay mixture is described in Table 2. The absorbance at 570 nm was compared to a methanol standard curve, and methanol in each sample was quantified.

TABLE 2
Composition of the reaction mixture for the methanol assay.

| | |
|---|---|
| Deionized water (mL) | 2.175 |
| 10x assay buffer (mL) | 0.25 |
| 100x Enzyme mixture (μL) | 25 |
| 50x colorimetric probe (μL) | 50 |
| Total reaction volume (mL) | 2.5 |

After raw absorbance data was collected, the data were processed as follows; background absorbance (media only) was subtracted from all samples, including the calibration samples. Each strain had been tested either with air injected or with methane injected. The absorbance from the air-injected sample was subtracted from the absorbance from the methane-injected sample. This absorbance value was compared with the calibration curve to determine the amount of methanol.

Some of the clones that were isolated by screening with the coumarin assay described above were subsequently selected for the methanol assay. Alongside these clones, a WT strain was assayed as a control. For each strain, the methanol titer was normalized by the OD600 (as a measure of the cell density) to measure the cellular productivity (methanol per cell). The data shown below in Table 3 has been normalized by this WT strain in order to show the improvements in methanol productivity, relative to the WT control as the starting point. Positions are numbered with respect to SEQ ID NO: 3.

TABLE 3
Improved mutants discovered by screening with methane
as a substrate and measuring the methanol titers
per OD600 relative to the WT mmoX gene.

| | | Titer/OD |
|---|---|---|
| | Incubation Time | (normalized |
| Mutation | (hours) | to WT) |
| G162C | 24.75 | 1.67 |
| R330T | 24.75 | 1.30 |

### Example 2: Mutagenesis of the Mutant Accessory Protein B

The method described above (in Example 1) was applied in an identical fashion to the mutagenesis of the mmoB gene in pBZ34 (SEQ ID NO: 1), with the exception of the saturation mutagenesis primers being targeted to the mmoB gene instead of the mmoX gene. Screening with coumarin as the substrate or methane as the substrate was performed as described above.

Results are shown below in tables 4-5, wherein positions are numbered with respect to SEQ ID NO: 6.

TABLE 4
Mutations in accessory
protein B with improved activity.

| Mutation(s) | Activity |
|---|---|
| V003G | 1.31 |
| L015M | 1.30 |
| K016G | 1.25 |
| V038G | 1.25 |
| T058E, | 1.24 |
| T118E | |
| N069K | 1.27 |
| A081G | 3.31 |
| N101G | 1.27 |
| V102A | 1.31 |
| V102L | 1.32 |
| Y103H | 2.02 |
| L106V | 1.27 |
| S110A | 1.40 |
| T112G | 1.50 |
| T112V | 1.30 |
| A116S | 1.54 |
| A116T | 1.49 |
| T118G | 1.34 |
| L119E | 1.26 |
| G120A | 1.83 |
| G120E | 1.58 |
| T124C | 1.31 |
| T124G | 1.37 |
| T126S | 1.44 |
| M130D | 1.25 |
| M130V | 1.44 |
| G131S | 1.39 |
| R134A | 1.53 |
| R134G | 1.38 |
| R134W | 1.37 |
| L136A | 1.21 |
| D138S | 1.29 |
| Y139G | 1.28 |
| Y139S | 1.30 |
| Y139V | 1.28 |

TABLE 5
Improved mutants discovered by screening with
methane as a substrate and measuring the methanol
titers per OD600 relative to the WT mmoB gene.

| | Incubation | Titer/OD |
|---|---|---|
| | Time | (normalized |
| Mutation | (hours) | to WT) |
| G120E | 18.50 | 1.07 |

### Example 3: Mutagenesis of Mutant Reductase Subunit C

The method described above (in Example 1) was applied in an identical fashion to the mutagenesis of the mmoC gene in pBZ34 (SEQ ID NO: 1), with the exception of the saturation mutagenesis primers being targeted to the mmoC gene instead of the mmoX gene.

Results are shown below in tables 6-7, wherein positions are numbered with respect to SEQ ID NO: 7.

TABLE 6
Mutations in reductase
subunit C with improved activity.

| Mutations | Activity |
|---|---|
| L017R, W240E | 2.71 |
| L017Q | 1.79 |
| I028G | 1.19 |
| L038G | 1.23 |
| E044R | 1.60 |
| E044G | 1.23 |
| G045R, S329G | 1.16 |
| T049L | 1.99 |
| D058K | 1.19 |
| K062L | 1.70 |
| C064Q | 1.17 |
| C064A, R330W | 1.15 |
| L069A | 1.30 |
| E074R | 1.41 |
| K087L, Q236M | 1.67 |
| L090Q, A216V | 1.17 |
| T097V | 1.17 |
| H098G | 1.41 |
| R100G, E288V | 1.11 |
| E110R, N308V | 1.31 |
| A111V | 1.21 |
| L116V, K211V | 1.27 |
| V119C | 1.10 |
| Q125L | 1.30 |
| D133V, D286T | 1.35 |
| C135T | 1.55 |
| R138Q, F208Q | 1.16 |
| E143K, P144C | 1.67 |
| I152S | 1.17 |
| P153H | 1.37 |
| T155A, C279S | 1.14 |
| V157R | 1.12 |
| E174A | 1.14 |
| L180S | 1.43 |
| R184G | 1.16 |
| N191D | 1.85 |
| G196V | 1.22 |
| P204S | 1.15 |
| G209W | 1.23 |
| R213G, W281R | 1.50 |
| A222G | 1.13 |
| R235G | 1.13 |
| Q238G | 1.14 |
| A242V | 1.57 |
| P243G, E245D | 1.12 |
| N244G | 1.14 |
| E245M | 1.68 |
| T246M | 1.12 |
| C324G | 1.39 |

TABLE 7
Improved mutants discovered by screening with methane
as a substrate and measuring the methanol titers per
OD600 relative to the WT mmoC gene.

| | Incubation | Titer/OD |
|---|---|---|
| | Time | (normalized |
| Mutation | (hours) | to WT) |
| Q125L | 21 | 1.81 |
| E174A | 21 | 1.75 |
| N168A | 21 | 1.74 |
| V140W | 16 | 1.25 |
| N308G | 16 | 1.19 |

### Example 4: Mutagenesis of the Hydroxylase Beta Subunit

The method described above (in Example 1) was applied in an identical fashion to the mutagenesis of the mmoY gene in pNH265 (SEQ ID NO: 2), with the exception of the saturation mutagenesis primers being targeted to the mmoY gene instead of the mmoX gene.

Results are shown below in tables 8-9, wherein positions are numbered with respect to SEQ ID NO: 4.

TABLE 8
Mutations in the hydroxylase
beta subunit with improved activity.

| Mutation(s) | Activity |
|---|---|
| N032V | 1.14 |
| G036L | 1.14 |
| L046V | 1.14 |
| T047H, | 1.10 |
| A151G | |
| T047V | 1.14 |
| P058N | 1.28 |
| A064G | 1.26 |
| T087V | 1.12 |
| V093G | 1.11 |
| Y109A | 1.14 |
| Y109E | 1.17 |
| Y109F | 1.11 |
| Y109V | 1.17 |
| I133S | 1.41 |
| E143T | 1.17 |
| N146R | 1.28 |
| N155G | 1.30 |
| Y157W | 1.31 |
| F160K | 1.15 |
| F160M | 1.21 |
| F160T | 1.40 |
| F160V | 1.35 |
| A162T, | 1.42 |
| K202Q | |
| A167Q | 1.25 |
| W182M | 1.12 |
| G183A | 1.31 |
| G183S | 1.18 |
| F184S | 1.14 |
| K186N | 1.28 |
| A201G, | 1.29 |
| G233R | |
| K202A | 1.55 |
| K202R, | 1.24 |
| A230G | |
| K202S | 1.43 |
| I203V, G273T | 1.62 |
| V204S | 1.27 |
| G206E | 1.50 |
| D208A, R318I | 1.33 |
| E209R | 2.33 |
| T211I | 1.37 |
| A212G, | 1.32 |
| A330R | |
| A212G | 2.79 |
| V213G | 1.48 |
| V213W | 1.26 |
| P214V | 1.29 |
| A216G, | 1.53 |
| G273V | |
| E217L | 1.30 |
| N220V | 1.77 |
| E222R, D305L | 1.36 |
| Y224M | 1.34 |
| K225G, | 1.99 |
| R333V | |
| S226G | 1.53 |
| G233L | 1.34 |
| G233W | 1.84 |
| Q236G | 1.61 |
| Q236V | 1.38 |
| Q236W | 1.48 |
| F239L | 1.60 |
| N242G | 1.29 |
| N242P, A291T | 1.76 |
| N242V | 1.48 |
| S244G, I281G | 1.35 |
| A250C, | 1.31 |
| L325G | |
| F256W | 1.25 |
| V260C | 1.44 |
| Q266V | 1.49 |
| A269G | 1.35 |
| P270R, D306Q | 1.35 |
| F272K | 1.24 |
| L276V | 1.54 |
| L303G | 2.58 |
| G304A | 1.22 |
| D305A | 1.38 |
| D305G | 1.58 |
| D305R | 1.74 |
| D305W | 1.62 |
| D306E | 1.61 |
| D306R | 1.66 |
| E308G, | 1.22 |
| R314H | |
| S310R | 1.66 |
| S310W | 1.75 |
| D311A | 1.39 |
| D311G | 1.83 |
| R314G | 1.26 |
| R314Q | 1.30 |
| R318G | 1.34 |
| G322R | 1.45 |
| T328G | 1.44 |
| I329W | 2.79 |
| F335G | 1.61 |
| M336G | 1.34 |
| L356G | 1.58 |
| I364G | 1.39 |
| D372W | 1.44 |

TABLE 9
Improved mutants discovered by screening with methane
as a substrate and measuring the methanol titers
per OD600 relative to the WT mmoY gene.

| | Incubation | Methanol Titer/OD |
|---|---|---|
| Mutation | Time (hours) | (normalized to WT) |
| N146R | 18 | 1.56 |
| G233W | 18 | 1.15 |
| D305R | 18 | 1.18 |
| A212G | 18 | 1.39 |
| R314G | 18.75 | 1.15 |
| I329W | 18 | 1.49 |

### Example 5: Mutagenesis of Hydroxylase Gamma Subunit

The method described above (in Example 1) was applied in an identical fashion to the mutagenesis of the mmoZ gene in pNH265 (SEQ ID NO: 2), with the exception of the saturation mutagenesis primers being targeted to the mmoZ gene instead of the mmoX gene.

Results are shown below in tables 10 and 11, wherein positions are numbered with respect to SEQ ID NO: 5.

TABLE 10
Mutations in hydroxylase gamma subunit with
improved activity (measured as fold-improvement
over unmutagenized sequence).

| Mutation(s) | Activity |
|---|---|
| D010R | 1.21 |
| D010V | 1.60 |
| N017F | 1.13 |
| N017W | 1.41 |
| K018A | 1.10 |
| K018E | 1.38 |
| A020G | 1.22 |
| Q021H, I112V | 1.38 |
| N023A | 1.11 |
| N023D | 1.11 |
| N023E | 1.32 |
| N023G | 1.35 |
| N023L | 1.21 |
| N023L, A117L | 1.54 |
| N023V | 1.22 |
| T024E | 1.24 |
| T024V, F043K | 1.42 |
| K027G | 1.33 |
| K027Q | 1.30 |
| K027R | 1.49 |
| A029G | 1.13 |
| A029G, E154G | 1.35 |
| M031W | 1.43 |
| K033V | 1.18 |
| Q034A | 1.19 |
| Q034K | 1.34 |
| T041G | 1.15 |
| F043S | 1.10 |
| F043V | 1.26 |
| N045G | 1.14 |
| S046G | 1.18 |
| S046I | 1.12 |
| Y047S | 1.37 |
| Y047T | 1.29 |
| D050G | 1.63 |
| N051G | 1.72 |
| N051V | 1.14 |
| D052G | 1.81 |
| Y053C | 1.66 |
| I056L | 1.31 |
| K063G | 1.23 |
| K063T | 1.13 |
| V066D | 1.33 |
| L067E | 1.31 |
| R070E | 1.77 |
| R070G | 1.11 |
| R070V | 1.34 |
| H079E | 1.28 |
| K080M | 1.27 |
| D103G, L166E | 1.15 |
| Q116S | 1.25 |
| Y127F | 1.29 |
| L129G | 1.18 |
| G136Q | 1.64 |
| G136S | 1.24 |
| N144D | 1.22 |
| N144G | 1.50 |
| N144V | 1.24 |
| Y147D | 1.30 |
| E153G | 1.46 |
| E154G | 1.42 |
| R156A | 1.13 |
| H165G | 1.40 |
| L166G | 1.29 |

TABLE 11
Improved mutants discovered by screening with methane
as a substrate and measuring the methanol titers per
OD600 relative to the Wild-type mmoZ gene.

| | Incubation | Methanol Titer/OD |
|---|---|---|
| Mutation | Time (hours) | (normalized to WT) |
| H165G | 20.25 | 1.07 |
| Y47S | 20.25 | 1.12 |
| Y47T | 18.75 | 1.35 |
| K27R | 18.75 | 1.20 |
| M31W | 18.75 | 1.23 |
| G136Q | 18.75 | 1.41 |
| A29G, | 18.75 | 1.40 |
| E154G | | |

### Example 6: Mutagenesis of mmoD

The method described above (in Example 1) was applied in an identical fashion to the mutagenesis of the mmoD gene in pNH265 (SEQ ID NO: 2), with the exception of the saturation mutagenesis primers being targeted to the mmoD gene instead of the mmoX gene.

Results are shown below in tables12 and 13, wherein positions are numbered with respect to SEQ ID NO: 8.

TABLE 12
Mutations in MMOD.
mmoD mutants

| | Normalized Titer |
|---|---|
| Mutation | Improvement |
| S4R | 1.17 |
| A5S | 1.16 |
| Q7G | 1.49 |
| P8G | 1.12 |
| F9G | 1.20 |
| S10R | 1.11 |
| A13E | 1.24 |
| D14R | 1.20 |
| F17G | 1.25 |
| E18R | 1.30 |
| P20G | 1.22 |
| R21L | 1.23 |
| P22R | 2.10 |
| A24G | 1.12 |
| F27V | 1.90 |
| T40R | 1.27 |
| E60Q | 2.70 |
| S74A | 1.15 |
| T79G | 1.39 |
| T79S | 1.71 |
| H80G | 1.25 |
| E89D | 1.39 |
| Q90S | 1.24 |
| A92P | 1.26 |
| P93A | 1.36 |
| D96R | 1.17 |

TABLE 13
Improved mutants discovered by screening with
methane as a substrate and measuring the methanol
titers per OD600 relative to the WT mmoD gene.

| | Incubation | Methanol Titer/OD |
|---|---|---|
| Mutation | Time (hours) | (normalized to WT) |
| E60Q | 18.5 | 1.47 |

### Example 7: Mutagenesis of GroEL-2

The method described above (in Example 1) was applied in an identical fashion to the mutagenesis of the groEL-2 gene in pNH265 (SEQ ID NO: 2), with the exception of the saturation mutagenesis primers being targeted to the groEL-2 gene instead of the mmoX gene.

Results are shown below in tables 14 and 15, wherein positions are numbered with respect to SEQ ID NO: 9.

TABLE 14
Mutations in groEL-2.
groEL-2 mutations

| | Normalized |
|---|---|
| | Titer |
| Mutation | Improvement |
| A109E | 1.43 |
| A109P | 1.94 |
| G160A | 1.75 |
| D168V | 1.39 |
| A169S | 2.50 |
| G171P | 1.05 |
| Q182W | 2.12 |
| L187V | 1.31 |
| T396L | 1.33 |
| V405G | 1.27 |
| V405D | 1.31 |
| N409G | 2.64 |
| A438K | 1.20 |
| D444S | 1.11 |
| P461V | 1.42 |
| G466L | 1.12 |
| A478L | 1.13 |
| A478R | 1.14 |
| A484E | 1.11 |
| A484G | 1.06 |
| N485T | 1.17 |
| G495H | 1.15 |
| V522L | 1.31 |
| G524T | 1.16 |
| T529G | 1.42 |

TABLE 15
Improved mutants discovered by screening with
methane as a substrate and measuring the methanol
titers per OD600 relative to the WT groEL-2 gene.

| | Incubation | Methanol Titer/OD |
|---|---|---|
| Mutation | Time (hours) | (normalized to WT) |
| N409G | 18.5 | 1.52 |

All publications and patent applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof.

1. A mutant soluble diiron monooxygenase system comprising at least one mutant subunit, wherein the at least one mutant subunit comprises one or more mutations, such that the activity of the mutant soluble diiron monooxygenase system is greater than the activity of a wild-type soluble diiron monooxygenase system.

2. The mutant soluble diiron monooxygenase system of claim 1, comprising a mutant hydroxylase alpha subunit wherein the mutant hydroxylase alpha subunit comprises one or more mutations at one or more residue position(s): 3, 14, 25, 26, 32, 33, 35, 42, 44, 53, 54, 55, 56, 57, 61, 130, 135, 154, 162, 163, 168, 216, 251, 262, 263, 264, 277, 316, 322, 330, 347, 356, 374, 375, 381, 387, 393, 399, 405, 419, 420, 422, 431, 432, 433, 434, 434, 444, 453, 456, 459, 469, 470, 475, 480, 485, 493, 504, 505, 510, 513, 515, 525, and/or 526, wherein the residue position(s) are numbered with respect to SEQ ID NO: 3.

3. The mutant soluble diiron monooxygenase hydroxylase alpha subunit according to claim 2, wherein the one or more mutations comprises substitutions at one or more of residue position(s): L3P, L14P, A25S, Q26G, L32P, Q33K, Q33R, F35L, N42R, T44A, T44S, A53G, N54K, N54L, E55A, E55S, T56A, T56G, K57G, K57S, K57T, K61A, K61R, A130C, A130G, A130H, A130T, N135S, V154L, G162C, Q163F, H168G, L216M, Y251M, Y251W, A262E, S263Q, A264Q, T277G, 1316S, G322N, R330T, Y347G, T356C, T356G, T356M, A374E, N375H, D381R, D381S, Y387I, Y387L, R393C, R399D, L405G, R419L, R419S, V420C, Q422E, K431E, G432A, A433G, A433Q, A433T, S434G, S434N, Q444E, G453V, M456V, A459E, I469V, F470I, G475*, E480G, L485E, K493E, K493V, D504E, K505E, D510E, K513R, K513T, L515G, A525Q, F526C, F526G, F526L, and/or F526S, wherein the residue position(s) are numbered with respect to SEQ ID NO: 3.

4. The mutant soluble diiron monooxygenase system of claim 1, comprising a mutant accessory protein subunit B wherein the mutant accessory protein subunit B comprises one or more mutations at one or more residue position(s): 3, 15, 16, 38, 58, 69, 81, 101, 102, 103, 106, 110, 112, 116, 118, 119, 120, 124, 126, 130, 131, 134, 136, 138, and/or 139, wherein the residue position(s) are numbered with respect to SEQ ID NO: 6.

5. The mutant soluble diiron monooxygenase accessory protein subunit B of claim 4, wherein the one or more mutations comprises substitutions at one or more residue position(s): V3G, L15M, K16G, V38G, T58E, N69K, A81G, N101G, V102A, V102L, Y103H, L106V, S110A, T112G, T112V, A116S, A116T, T118E, T118G, L119E, G120A, G120E, T124C, T124G, T126S, M130D, M130V, G131S, R134A, R134G, R134W, L136A, D138S, Y139G, Y139S, and/or Y139V, wherein the residue position(s) are numbered with respect to SEQ ID NO: 6.

6. The mutant soluble diiron monooxygenase system of claim 1, comprising a mutant reductase subunit C wherein the mutant reductase subunit C comprises one or more mutations at one or more residue position(s): 17, 28, 38, 44, 45, 49, 58, 62, 64, 69, 74, 87, 90, 97, 98, 100, 110, 111, 116, 119, 125, 133, 135, 138, 143, 144, 152, 153, 155, 157, 174, 180, 184, 191, 196, 204, 208, 209, 211, 213, 216, 222, 235, 236, 238, 240, 242, 243, 244, 245, 246, 279, 281, 286, 288, 308, 324, 329, and/or 330, wherein the residue position(s) are numbered with respect to SEQ ID NO: 7.

7. The mutant soluble diiron monooxygenase reductase subunit C of claim 6, wherein the one or more mutations comprises substitutions at one or more residue position(s): L17R, L17Q, I28G, L38G, E44G, E44R, G45R, T49L, D58K, K62L, C64A, C64Q, L69A, E74R, K87L, L90Q, T97V, H98G, R100G, E110R, A111V, L116V, V119C, Q125L, D133V, C135T, R138Q, E143K, P144C, I152S, P153H, T155A, V157R, E174A, L180S, R184G, N191D, G196V, P204S, F208Q, G209W, K211V, R213G, A216V, A222G, R235G, Q236M, Q238G, W240E, A242V, P243G, N244G, E245D, E245M, T246M, C279S, W281R, D286T, E288V, N308V, C324G, S329G, and/or R330W, wherein the residue position(s) are numbered with respect to SEQ ID NO: 7.

8. The mutant soluble diiron monooxygenase system of claim 1, comprising a mutant hydroxylase beta subunit wherein the mutant hydroxylase beta subunit comprises one or more mutations at one or more residue position(s): 32, 36, 46, 47, 58, 64, 87, 93, 109, 133, 143, 146, 151, 155, 157, 160, 162, 202, 167, 182, 183, 184, 186, 201, 202, 203, 204, 206, 208, 209, 211, 212, 213, 214, 216, 217, 220, 222, 224, 225, 226, 230, 233, 236, 239, 242, 244, 250, 256, 260, 266, 269, 270, 272, 273, 276, 281, 291, 303, 304, 305, 306, 308, 310, 311, 314, 318, 322, 325, 328, 329, 330, 333, 335, 336, 356, 364, and/or 372, wherein the residue position(s) are numbered with respect to SEQ ID NO: 4.

9. The mutant soluble diiron monooxygenase hydroxylase beta subunit of claim 8, wherein the one or more mutations comprises substitutions at one or more residue position(s): N32V, G36L, L46V, T47H, T47V, P58N, A64G, T87V, V93G, Y109A, Y109E, Y109F, Y109V, I133S, E143T, N146R, A151G, N155G, Y157W, F160K, F160M, F160T, F160V, A162T, 202, A167Q, W182M, G183A, G183S, F184S, K186N, A201G, K202A, K202Q, K202R, K202S, I203V, V204S, G206E, D208A, E209R, T211I, A212G, V213G, V213W, P214V, A216G, E217L, N220V, E222R, Y224M, K225G, S226G, A230G, G233L, G233R, G233W, Q236G, Q236V, Q236W, F239L, N242G, N242P, N242V, S244G, A250C, F256W, V260C, Q266V, A269G, P270R, F272K, G273T, G273V, L276V, I281G, A291T, L303G, G304A, D305A, D305G, D305L, D305R, D305W, D306E, D306Q, D306R, E308G, S310R, S310W, D311A, D311G, R314G, R314H, R314Q, R318G, R318I, G322R, L325G, T328G, 1329W, A330R, R333V, F335G, M336G, L356G, 1364G, and/or D372W, wherein the residue position(s) are numbered with respect to SEQ ID NO: 4.

10. The mutant soluble diiron monooxygenase system, according to claim 1, comprising a mutant hydroxylase gamma subunit wherein the mutant hydroxylase gamma subunit comprises one or more mutations at one or more residue position(s): 10, 17, 18, 20, 21, 23, 24, 27, 29, 31, 33, 34, 41, 43, 45, 46, 47, 50, 51, 52, 53, 56, 63, 66, 67, 70, 79, 80, 103, 112, 116, 117, 127, 129, 136, 144, 147, 153, 154, 156, 165, and/or 166, wherein the residue position(s) are numbered with respect to SEQ ID NO: 5.

11. The mutant soluble diiron monooxygenase hydroxylase gamma subunit of claim 10, wherein the one or more mutations comprises substitutions at one or more residue position(s): D10R, D10V, N17F, N17W, K18A, K18E, A20G, Q21H, N23A, N23D, N23E, N23G, N23L, N23V, T24E, T24V, K27G, K27Q, K27R, A29G, M31W, K33V, Q34A, Q34K, T41G, F43K, F43S, F43V, N45G, S46G, S46I, Y47S, Y47T, D50G, N51G, N51V, D52G, Y53C, I56L, K63G, K63T, V66D, L67E, R70E, R70G, R70V, H79E, K80M, D103G, 1112V, Q116S, 1117L, Y127F, L129G, G136Q, G136S, N144D, N144G, N144V, Y147D, E153G, E154G, R156A, H165G, L166E, and/or L166G, wherein the residue position(s) are numbered with respect to SEQ ID NO: 5.

12. The mutant soluble diiron monooxygenase system of claim 1, comprising a mutant subunit D wherein the mutant subunit D comprises one or more mutations at one or more residue position(s): 4, 5, 7, 8, 9, 10, 13, 14, 17, 18, 20, 21, 22, 24, 27, 40, 60, 74, 79, 80, 89, 90, 92, 93, and/or 96, wherein the residue position(s) are numbered with respect to SEQ ID NO: 8.

13. The mutant soluble diiron monooxygenase subunit D of claim 12, wherein the one or more mutations comprises substitutions at one or more residue position(s): S4R, A5S, Q7G, P8G, F9G, F9G, S10R, A13E, D14R, F17G, F17G, E18R, P20G, R21L, P22R, A24G, F27V, T40R, E60Q, S74A, T79G, T79S, H80G, E89D, Q90S, A92P, P93A, and/or D96R, wherein the residue position(s) are numbered with respect to SEQ ID NO: 8.

14. The mutant soluble diiron monooxygenase system of claim 1, comprising a mutant protein folding chaperone groEL-2 wherein the mutant protein folding chaperone groEL-2 comprises one or more mutations at one or more residue position(s): 109, 160, 168, 169, 171, 182, 187, 396, 405, 409, 438, 444, 461, 466, 478, 484, 485, 495, 522, 524, and/or 529, wherein the residue position(s) are numbered with respect to SEQ ID NO: 9.

15. The mutant protein folding chaperone groEL-2 of claim 14, wherein the one or more mutations comprises substitutions at one or more residue position(s): A109E, A109P, G160A, D168V, A169S, G171P, Q182W, L187V, T396L, V405G, V405D, N409G, A438K, D444S, P461V, G466L, A478L, A478R, A484E, A484G, N485T, G495H, V522L, G524T, and/or T529G, wherein the residue position(s) are numbered with respect to SEQ ID NO: 9.

16. One or more isolated nucleic acids encoding one or more polypeptides comprising or consisting of the mutant soluble diiron monooxygenase system of claim 1.

17. One or more vectors comprising or consisting of the one or more isolated nucleic acids of claim 16.

18. One or more host cells comprising or consisting of the one or more vectors of claim 17.

19. The one or more host cells of claim 18, wherein the one or more host cells comprises a prokaryotic cell.

20. The one or more host cells of claim 19, wherein the prokaryotic cell comprises one or more of Escherichia coli, Corynebacterium glutamicum, and/or Bacillus methanolicus.
